(19) United States
(12) Patent Application Publication
(10) Pub. No.: US 2026/0335158 A1
(43) Pub. Date: Oct. 29, 2026

(54) CONTROLS FOR DUAL COLUMN SPINE SURGERY TABLE

(71) Applicant: Allen Medical Systems, Inc., Batesville, IN (US)

(72) Inventors: David J. Hitchcock, Westford, MA (US); Timothy M. Volk, Harrison, OH (US); Bryston Cotton, Aurora, IN (US); Andrew R. Marchand, North Billerica, MA (US)

(21) Appl. No.: 19/467,687

(22) Filed: Feb. 2, 2026

Related U.S. Application Data
(60) Provisional application No. 63/758,668, filed on Feb. 14, 2025.

(51) Int. Cl. A61G 13/00; A61G 13/06; A61G 13/08; A61G 13/10

(52) U.S. Cl. CPC A61G 13/0054; A61G 13/06; A61G 13/08; A61G 13/104

(57) ABSTRACT

A patient support apparatus includes a support base and a lift system that includes a first lift column coupled with the support base and a second lift column spaced apart from the first lift column and coupled with support base. The lift system is configured to support a patient support above a floor underlying the patient support apparatus and to rotate the patient support about an axis that extends through the first lift column and the second lift column.

### CROSS-REFERENCE TO RELATED APPLICATIONS

This patent application claims priority to U.S. Provisional Patent Application No. 63/758,668 which was filed Feb. 14, 2025 and which is hereby incorporated by reference herein in its entirety.

### BACKGROUND

The present disclosure is directed to patient support apparatuses such as are used to support a patient in different positions. More specifically, the present disclosure is related to surgical tables for use in positioning and re-positioning a patient during spinal surgery, for example, to provide for adjustability in the coronal plane. However, the present disclosure may also be applicable to other types of patient support apparatuses such as hospital beds, home care beds, x-ray tables, therapy supports, wheel chairs, and the like.

Conventional patient support apparatuses may be configured to rotate, tilt, and/or raise and lower a frame or patient support relative to a base. Such movements allow a surgeon or other medical provider to more easily access desired areas of the patient located on the patient support. The movement of the patient support apparatus may be controlled through a remote pendant or other user interface and the status of the position of the patient support apparatus may be indicated with status lights on the remote pendant or a panel on the patient support apparatus. There remains a desire to provide additional interfaces for controlling operation of the patient support apparatus and for providing indicia to allow the user to quickly understand the position and status of the patient support apparatus by looking at the indicators on or near the patient support apparatus controls.

Moreover, patient support apparatuses may include casters for moving the patient support apparatus within a hospital facility. Each caster includes a wheel and a braking element that is movable between a braked and an unbraked position to block or allow the wheel to roll and to facilitate movement of the patient support apparatus. Some patient support apparatuses include controls and actuation features for electronically braking and unbraking the braking elements of the casters. However, if the patient support apparatus is without electric power, it is desirable to provide a manual option for operating the braking elements. As such, there remains a need for braking systems that are electrically operated when electric power is available and while allowing an option for manual release of the braking elements, for example, if power is unavailable.

### SUMMARY

The present disclosure includes one or more of the features recited in the appended claims and/or the following features which, alone or in any combination, may comprise patentable subject matter.

According to a first aspect of the present disclosure, a patient support apparatus includes a support base; a first lift column and a second lift column supported by the support base, and a patient support. The second lift column is spaced apart from the first lift column. The patient support extends between and is coupled with the first lift column and the second lift column. The patient support is rotatable about an axis that extends between the first lift column and the second lift column.

In some embodiments of the patient support apparatus of the first aspect, a height of the first lift column and the height of the second lift column are adjustable to locate the axis in a horizontal orientation above a floor underlying the support base by a vertical distance. In some embodiments, the vertical distance may be equal to or less than 900 millimeters.

In some embodiments of the patient support apparatus of the first aspect, the first lift column may include at least three telescoping segments and an actuator configured to adjust a height of the first lift column. The second lift column may include at least three telescoping segments and an actuator configured to adjust a height of the second lift column.

In some embodiments of the patient support apparatus of the first aspect, the height of the first lift column and the height of the second lift column may be adjustable relative to the support base to angle the axis to be non-parallel with the floor.

In some embodiments of the patient support apparatus of the first aspect, the height of the first lift column and the height of the second lift column may be adjustable to locate the axis in the horizontal orientation above the floor with the vertical distance being 800 millimeters or less. In some embodiments of the patient support apparatus of the first aspect, the height of the first lift column and the height of the second lift column may be adjustable to locate the axis in the horizontal orientation above the floor underlying the base with the vertical distance being equal to 1,200 millimeters or more.

In some embodiments of the patient support apparatus of the first aspect, the height of the first lift column and the height of the second lift column may be adjustable such that a ratio between a highest value of the vertical distance and a lowest value of the vertical distance may be greater than or equal to 1.5:1. In some embodiments of the patient support apparatus of the first aspect, the ratio may be greater than or equal to 1.6:1.

In some embodiments of the patient support apparatus of the first aspect, the support base may have a top surface spaced apart vertically from the floor by a height equal to 250 millimeters or less. In some embodiments of the patient support apparatus of the first aspect, the height of the first lift column and the height of the second lift column may be adjustable relative to the base such that a ratio between the height of the axis in the horizontal orientation from the floor and the height of the base from the floor may be variable between at least 3.5:1 and 5:1.

In some embodiments of the patient support apparatus of the first aspect, the patient support apparatus may further include a caster coupled with the base and an electric actuator. The caster may be configured to move the patient support apparatus relative to the floor and the electric actuator may be coupled with the base and configured to move the caster between an unbraked position to a braked position.

In some embodiments of the patient support apparatus of the first aspect, the patient support apparatus may include a light system. The light system may be configured to display a light indicating the caster is in either the braked position or the unbraked position.

In some embodiments of the patient support apparatus of the first aspect, the light system may be configured to illuminate the floor with the light. In some embodiments of the patient support apparatus of the first aspect, the light may be a first color in response to the caster being in the unbraked position and the light may be a second color different from the first color in response to the caster being in the braked position.

In some embodiments of the patient support apparatus of the first aspect, the patient support apparatus may further include an accessory rail coupled with the first column. In some embodiments of the patient support apparatus of the first aspect, the accessory rail may include a first rail body extending laterally away from the first lift column. The first rail body may be formed to define a first channel extending posteriorly partway into the first rail body and a second channel extending anteriorly partway into the first rail body.

In some embodiments of the patient support apparatus of the first aspect, the accessory rail may include a second rail body that extends anteriorly and posteriorly away from an end of the first rail body. In some embodiments of the patient support apparatus of the first aspect, the first rail body may be formed to include an elongated slot that extends through the first rail body and opens into the first channel and the second channel.

In some embodiments of the patient support apparatus of the first aspect, the patient support apparatus may further include an extension. The extension may extend between and interconnect a first base and a second base included in the support base for moving the support base between a deployed position in which the first base is spaced apart from the second base by a first distance and a collapsed position in which the first base is spaced apart from the second base by a second distance that is less than the first distance.

In some embodiments of the patient support apparatus of the first aspect, the extension may include a first tube coupled with the first base, a second tube coupled with the second base and arranged within the first tube to allow telescoping movement of the first tube and the second tube, and a lock unit. The lock unit may be configured to move between a locked arrangement in which relative movement between the first tube and the second tube is blocked and an unlocked arrangement in which relative movement between the first tube and the second tube is allowed to move the support base between the collapsed position and the deployed position.

In some embodiments of the patient support apparatus of the first aspect, the first tube may include a tube body and a band coupled with the tube body. The lock unit may be configured to move selectively the band relative to the tube body of the first tube to apply a clamping force to the second tube and block relative movement between the first tube and the second tube. In some embodiments of the patient support apparatus of the first aspect, the tube body and the band of the first tube may be integrally formed.

In some embodiments of the patient support apparatus of the first aspect, the lock unit may include a first mount plate coupled with the tube body, a second mount plate coupled with a free end of the band, and a pedal assembly. The pedal assembly may be configured to move, selectively, the second mount plate relative to the first mount plate to cause the band to apply the clamping force to the second tube.

In some embodiments of the patient support apparatus of the first aspect, the pedal assembly may include a pin, a cam body, and a foot lever. The pin may be coupled with the second mount plate. The cam body may be pivotably coupled with the pin and engaged with the first mount plate. The foot lever may be coupled with the cam body and configured to rotate the cam body relative to the first mount plate to cause the pin to move the second plate toward and away from the first mount plate.

In some embodiments of the patient support apparatus of the first aspect, the patient support apparatus may further include a rotation indicator coupled with the first lift column. The rotation indicator may be configured to display information indicative of an angle of rotation of the patient support about the axis. The rotation indicator may include a gauge track and a pointer that moves relative to the gauge track in response to rotation of the patient support about the axis.

In some embodiments of the patient support apparatus of the first aspect, the gauge track may be formed to define a pointer display opening that exposes the pointer to a user and a guide slot. The pointer may include a pointer body that extends into the guide slot to slide along the guide slot and a marker coupled with the pointer body and arranged such that at least a portion of the marker is visible to the user through the pointer display opening.

In some embodiments of the patient support apparatus of the first aspect, the rotation indicator may include a slide lever configured to rotate about the axis. The slide lever may be engaged with the pointer to cause the pointer to move in the guide slot in response to rotation of the patient support about the axis to indicate to the user the angle of rotation of the patient support apparatus about the axis.

In some embodiments of the patient support apparatus of the first aspect, the slide lever may extend in a cantilever relationship from a first end coupled with the first lift column. The slide lever may be configured to rotate in response to rotation of the patient support to a second end that extends into the pointer body such that pointer body is free to slide along the slide lever as the slide lever urges the pointer body to move in the gauge slot.

In some embodiments of the patient support apparatus of the first aspect, the rotation indicator may be coupled to a top face of the first lift column. The gauge track and marker may be visible to a user viewing down at the top face of the first lift column.

In some embodiments of the patient support apparatus of the first aspect, the patient support apparatus may include a controller including a processor and a memory device. The memory device may include instructions that are executable by the processor to cause the first lift column and the second lift column to move from a first arrangement in which a first shaft of the first lift column coupled with the patient support is at a first height and a second shaft of the second lift column coupled with the patient support is at a second height different from the first height to a second arrangement in which the first shaft is at a third height and the second shaft is at the third height.

In some embodiments of the patient support apparatus of the first aspect, the instructions, when executed by the processor, may cause the first lift column to move the first shaft from the first height to the third height and the second lift column to move the second shaft from the second height to the third height simultaneously.

In some embodiments of the patient support apparatus of the first aspect, the third height is between the first height and the second height. In some embodiments of the patient support apparatus of the first aspect, the third height is about halfway between the first height and the second height.

In some embodiments of the patient support apparatus of the first aspect, the instructions, when executed by the processor, may further cause the first lift column and the second lift column to rotate the patient support about the axis to a predetermined orientation relative to the lift system. In some embodiments of the patient support apparatus of the first aspect, the predetermined orientation may be a zero degrees position in which the patient support is configured to orient a patient supported thereon in a face up or face down arrangement.

According to a second aspect of the present disclosure, a patient support apparatus includes a support base, a lift system, and an extension. The support base includes a first base and a second base spaced apart from the first base. The lift system includes a first lift column coupled with the first base and a second lift column coupled with the second base. The first lift column and the second lift column are configured to raise and lower, independently, relative to the support base. The extension extends between and interconnects the first base and the second base. The extension is configured for moving the support base between a deployed position in which the first base is spaced apart from the second base by a first distance and a collapsed position in which the first base is spaced apart from the second base by a second distance that is less than the first distance.

In some embodiments of the patient support apparatus of the second aspect, the extension includes a first tube coupled with the first base, a second tube coupled with the second base and arranged within the first tube to allow telescoping movement of the first tube and the second tube, and a lock unit. The lock unit may be configured to move between a locked arrangement in which relative movement between the first tube and the second tube is blocked and an unlocked arrangement in which relative movement between the first tube and the second tube is allowed to move the support base between the collapsed position and the deployed position.

In some embodiments of the patient support apparatus of the second aspect, the first tube may include a tube body and a band coupled with the tube body. The lock unit may be configured to move selectively the band relative to the tube body of the first tube to apply a clamping force to the second tube and block relative movement between the first tube and the second tube. In some embodiments of the patient support apparatus of the second aspect, the tube body and the band of the first tube are integrally formed.

In some embodiments of the patient support apparatus of the second aspect, the lock unit may include a first mount plate coupled with the tube body, a second mount plate coupled with a free end of the band, and a pedal assembly. The pedal assembly maybe configured to move, selectively, the second mount plate relative to the first mount plate to cause the band to apply the clamping force to the second tube.

In some embodiments of the patient support apparatus of the second aspect, the pedal assembly may include a pin coupled with the second mount plate, a cam body pivotably coupled with the pin and engaged with the first mount plate, and a foot lever coupled with the cam body. The foot lever may be configured to rotate the cam body relative to the first mount plate to cause the pin to move the second plate toward and away from the first mount plate.

In some embodiments of the patient support apparatus of the second aspect, the patient support apparatus may include a patient support. The patient support may extend between and be coupled with the first lift column and the second lift column. The patient support may be rotatable about an axis that extends between the first lift column and the second lift column.

According to a third aspect of the present disclosure, a patient support apparatus includes a support base, a lift system, and an accessory rail. The support base includes a first base and a second base spaced apart from the first base. The lift system includes a first lift column coupled with the first base and a second lift column coupled with the second base. The first lift column and the second lift column are configured to raise and lower, independently, relative to the support base. The accessory rail is coupled to the first lift column and configured to support an accessory component removably coupled with the accessory rail.

In some embodiments of the patient support apparatus of the third aspect, the accessory rail may include a first rail body extending laterally away from the first lift column. The first rail body may be formed to define a first channel extending posteriorly partway into the first rail body and a second channel extending anteriorly partway into the first rail body.

In some embodiments of the patient support apparatus of the third aspect, the accessory rail may include a second rail body. The second rail body may extend anteriorly and posteriorly away from an end of the first rail body.

In some embodiments of the patient support apparatus of the third aspect, the first rail body may be formed to include an elongated slot. The elongated slot may extend through the first rail body and open into the first channel and the second channel.

In some embodiments of the patient support apparatus of the third aspect, the first lift column may include a first telescoping segment and a second telescoping segment. The first telescoping segment may be coupled with the first base. The accessory rail may be coupled to a side of the second telescoping segment.

In some embodiments of the patient support apparatus of the third aspect, the patient support apparatus may include a patient support extends between and is coupled with the first lift column and the second lift column. The patient support may be rotatable about an axis that extends between the first lift column and the second lift column.

According to a fourth aspect of the present disclosure, a patient support apparatus includes a support base, a lift system, and a caster brake status indicator system. The support base includes a first base and a second base spaced apart from the first base. The first base includes a caster having a wheel and a braking element and an electric actuator operably connected with the braking element of the caster to move the braking element between a braked position to block rotation of the wheel and an unbraked position to allow rotation of the wheel. The lift system includes a first lift column coupled with the first base and a second lift column coupled with the second base. The first lift column and the second lift column are configured to raise and lower, independently, relative to the support base. The caster brake status indicator system illuminates a light indicative of the braking element being in the unbraked position or the braked position.

In some embodiments of the patient support apparatus of the fourth aspect, the caster brake status indicator system may illuminate the light onto a floor underlying the patient support apparatus. In some embodiments of the patient support apparatus of the fourth aspect, the light may be a first color in response to the braking system being in the unbraked position. The light may be a second color different from the first color in response to the braking system being in the braked position.

According to a fifth aspect of the present disclosure, a patient support apparatus includes a support base, a lift system, a patient support, and a rotation indicator. The lift system includes a first lift column coupled with the support base and a second lift column spaced apart from the first lift column and coupled with the support base. The first lift column and the second lift column are configured to raise and lower, independently, relative to the support base. The patient support extends between and is coupled with the first lift column and the second lift column for vertical movement therewith. The patient support is rotatable about an axis that extends between the first lift column and the second lift column. The rotation indicator is coupled with the first lift column and configured to display information indicative of an angle of rotation of the patient support about the axis. The rotation indicator includes a gauge track and a pointer that moves relative to the gauge track in response to rotation of the patient support about the axis.

In some embodiments of the patient support apparatus of the fifth aspect, the gauge track may be formed to define a pointer display opening that exposes the pointer to a user and a guide slot. The pointer may include a pointer body that extends into the guide slot to slide along the guide slot and a marker coupled with the pointer body and arranged such that at least a portion of the marker is visible to the user through the pointer display opening.

In some embodiments of the patient support apparatus of the fifth aspect, the rotation indicator may include a slide lever configured to rotate about the axis. The slide lever may be engaged with the pointer to cause the pointer to move in the guide slot in response to rotation of the patient support about the axis to indicate to the user the angle of rotation of the patient support apparatus about the axis.

In some embodiments of the patient support apparatus of the fifth aspect, the slide lever may extend in a cantilever relationship from a first end to a second end. The first end may be coupled with the first lift column and configured to rotate in response to rotation of the patient support. The second end may extend into the pointer body such that pointer body is free to slide along the slide lever as the slide lever urges the pointer body to move in the gauge slot.

In some embodiments of the patient support apparatus of the fifth aspect, the rotation indicator may be coupled to a top face of the first lift column. The gauge track and the marker may be visible to a user viewing down at the top face of the first lift column.

According to a sixth aspect of the present disclosure, a patient support apparatus includes a support base, a lift system, a patient support, and a controller. The support base includes a first base and a second base spaced apart from the first base. The lift system includes a first lift column coupled with the first base and a second lift column coupled with the second base. The first lift column and the second lift column are configured to raise and lower, independently, relative to the support base. The patient support extends between and is coupled with a first shaft of the first lift column and a second shaft of the second lift column for vertical movement therewith. The patient support is rotatable about an axis that extends through the first shaft of the first lift column and the second shaft of the second lift column. The controller includes a processor and a memory device. The memory device includes instructions that are executable by the processor to cause the first lift column and the second lift column to move from a first arrangement in which the first shaft is at a first height and the second shaft is at a second height different from the first height to a second arrangement in which the first shaft is at a third height and the second shaft is at the third height.

In some embodiments of the patient support apparatus of the sixth aspect, the third height may be between the first height and the second height. In some embodiments of the patient support apparatus of the sixth aspect, the third height may be about halfway between the first height and the second height.

In some embodiments of the patient support apparatus of the sixth aspect, the instructions, when executed by the processor, may cause the first lift column and the second lift column to rotate the patient support about the axis to a predetermined orientation relative to the lift system. In some embodiments of the patient support apparatus of the sixth aspect, the predetermined orientation may be a zero degrees position in which the patient support is configured to orient a patient supported thereon in a face up or face down arrangement.

In some embodiments of the patient support apparatus of the sixth aspect, the instructions, when executed by the processor, may cause the first lift column to move the first shaft from the first height to the third height and the second lift column to move the second shaft from the second height to the third height simultaneously.

According to a seventh aspect of the present disclosure, a method of operating a patient support apparatus includes moving a first lift column to a first height relative to a support base coupled with the first lift column; moving a second lift column to a second height different from the first height relative to the support base coupled with the second lift column to tilt a patient support coupled with the first lift column and the second lift column relative to a floor; moving each of the first lift column and the second lift column to a third height different from the first height and the second height simultaneously to level the patient support relative to the floor.

In some embodiments of the method of the seventh aspect, the method includes rotating the patient support about an axis that extends through the first lift column and the second lift column from a first angle to a second angle different from the first angle. In some embodiments of the patient support apparatus of the seventh aspect, the method may include rotating the patient support from the second angle to a third angle corresponding with a zero degrees position in which the patient support is configured to orient a patient supported thereon in a face up or face down arrangement.

In some embodiments of the method of the seventh aspect, rotating the patient support from the second angle to the third angle may be performed simultaneously with moving each of the first lift column and the second lift column to the third height.

According to an eighth aspect of the present disclosure, a patient support apparatus includes a caster, a brake drive, and a linkage. The caster includes a stem, a wheel coupled to the stem to rotate about a wheel axis relative to the stem, and a braking element. The braking element is movable from an unbraked position allowing rotation of the wheel relative to the stem to a braked position blocking the wheel from rotation relative to the stem. The brake drive includes a mount and a linear actuator coupled to the mount and movable between a retracted position and an extended position relative to the mount. The linkage is configured to transfer motion of the linear actuator to the braking element to move the braking element between the unbraked position and the braked position. The linkage includes a shaft assembly, a clutch assembly, and a manual release assembly. The shaft assembly is coupled with the braking element. The clutch assembly is configured to be rotated about a clutch axis relative to the mount by the linear actuator to move the braking element between the unbraked position and the braked position via the shaft assembly. The manual release assembly is coupled with the clutch assembly and the shaft assembly and configured to operably decouple the linear actuator from the shaft assembly and bias the shaft assembly into moving the braking element to the unbraked position in response to a user manually activating the manual release.

In some embodiments of the patient support apparatus of the eighth aspect, the clutch assembly may be movable between an engaged arrangement and a disengaged arrangement. In the engaged arrangement, the clutch is rotatable by the linear actuator to transmit motion from the linear actuator through the shaft assembly to the braking element to move the braking element between the unbraked position and the braked position. In the disengaged arrangement, the shaft assembly is operably decoupled from the linear actuator.

In some embodiments of the patient support apparatus of the eighth aspect, the clutch assembly may include a clutch shaft, a first clutch plate, and a second clutch plate. The clutch shaft may be rotatable about the clutch axis and coupled to the shaft assembly for movement therewith. The first clutch plate may be coupled with the linear actuator and arranged to rotate selectively about the clutch axis relative to the clutch shaft. The second clutch plate may be coupled for rotation to the clutch shaft and movable between an engaged position and a disengaged position. In the engaged position, the second clutch plate is rotatably coupled with the first clutch plate such that the clutch assembly transmits motion from the linear actuator through the first clutch plate, the second clutch plate, and the clutch shaft to the shaft assembly. In the disengaged position, the second clutch plate is rotatably decoupled from the first clutch plate such that the second clutch plate and the shaft assembly are free to move relative to the first clutch plate and the linear actuator.

In some embodiments of the patient support apparatus of the eighth aspect, the second clutch plate may be movable axially along the clutch axis relative to the clutch shaft to move between the engaged position and the disengaged position.

In some embodiments of the patient support apparatus of the eighth aspect, one of the first clutch plate and the second clutch plate may be formed to define a recess therein. The other of the first clutch plate and the second clutch plate may be formed to include a tab that extend into the recess to rotatably couple the first clutch plate with the second clutch plate in response to the second clutch plate being in the engaged position and that is spaced from the recess to rotatably decouple the second clutch plate from the first clutch plate in response to the second clutch plate being in the disengaged position.

In some embodiments of the patient support apparatus of the eighth aspect, the clutch further may include a key. The key may interlock the second clutch plate with the clutch shaft while allowing the second clutch plate to slide axially relative to the clutch shaft.

In some embodiments of the patient support apparatus of the eighth aspect, the manual release assembly may include a plunger that is movable relative to the mount, a clutch release fork coupled with the clutch assembly, and a clutch release linkage. The clutch release linkage may be coupled with the plunger and the clutch release fork and may be configured to cause the clutch release fork to move the clutch assembly and operably decouple the linear actuator from the shaft assembly in response to the user pulling the plunger.

In some embodiments of the patient support apparatus of the eighth aspect, the clutch release fork may be coupled with the second clutch plate. The clutch release fork may be configured to move selectively the second clutch plate axially away from the first clutch plate to rotatably decouple the first clutch plate from the second clutch plate in response to the user pulling the plunger.

In some embodiments of the patient support apparatus of the eighth aspect, the clutch release fork may be biased to urge the second clutch plate into engagement with the first clutch plate.

In some embodiments of the patient support apparatus of the eighth aspect, the clutch release linkage may include a first lever and a second lever. The first lever may be pivotably coupled with the plunger and rotatable about a first lever axis. The second lever may be pivotably coupled with the first lever and the clutch release fork and rotatable about a second lever axis such that pulling the plunger causes the first lever to rotate the second lever about the second lever axis and move the clutch release fork and the second clutch plate away from the first clutch plate.

In some embodiments of the patient support apparatus of the eighth aspect, the first lever may be formed to include an elongated slot and the second lever may include a lever body and a pin that extends through the lever body and the elongated slot to couple the second lever with the first lever. The elongated slot may allow for limited movement of the first lever relative to the second lever to allow the plunger to be returned toward an initial position after being pulled while the second clutch plate is disengaged with the first clutch plate.

In some embodiments of the patient support apparatus of the eighth aspect, the manual release assembly may include a brake-release lever assembly coupled with the shaft assembly and a base of the patient support apparatus. The brake-release lever may be configured to urge the shaft assembly into moving the braking element toward the unbraked position.

In some embodiments of the patient support apparatus of the eighth aspect, the brake-release lever assembly may include a first brake-release lever and a first bias member. The first brake-release lever may be pivotably coupled with the base and with the shaft assembly and configured to rotate about a first brake-release lever axis. The first bias member may be coupled with the first brake-release lever and the base to bias the first brake-release lever such that the first brake-release lever urges the shaft assembly into moving the braking element toward the unbraked position.

In some embodiments of the patient support apparatus of the eighth aspect, the brake-release lever assembly may include a second brake-release lever and a second bias member. The second brake-release lever may be pivotably coupled with the base to rotate about a second brake-release lever axis and coupled with the first brake-release lever. The second bias member may be coupled with the base and with the second brake-release lever to cause the second brake-release lever to dampen movement of the first brake-release lever.

In some embodiments of the patient support apparatus of the eighth aspect, the first brake-release lever may include a first curved elongated slot and the second brake-release lever may include a second curved elongated slot. The brake-release lever assembly may include a first pin extending through the first curved elongated slot and the second brake-release lever and a second pin extending through the second curved elongated slot and into the base.

In some embodiments of the patient support apparatus of the eighth aspect, the patient support apparatus includes a second caster having a second wheel and a second braking element movable between an unbraked position and a braked position. The shaft assembly may include a first caster brake link coupled with the first braking element, a second caster brake link coupled with the second braking element and operably coupled with the linear actuator, and a cam coupled with one of the first caster brake link and the second caster brake link. The first caster brake link may be movable selectively relative to the second caster brake link. The cam may be configured to engage the other of the first caster brake link and the second caster brake link to urge selectively the first caster brake link away from the second caster brake link.

In some embodiments of the patient support apparatus of the eighth aspect, the patient support apparatus may include a caster brake status indicator system. The caster brake status indicator system may illuminate a light indicative of the braking element being in the unbraked position or the braked position.

In some embodiments of the patient support apparatus of the eighth aspect, the caster brake status indicator system may illuminate the light onto a floor underlying the patient support apparatus. In some embodiments of the patient support apparatus of the eighth aspect, the light may be a first color in response to the braking system being in the unbraked position and the light may be a second color different from the first color in response to the braking system being in the braked position.

In some embodiments of the patient support apparatus of the eighth aspect, the brake drive may include a travel stop. The travel stop may be fixed relative to the mount and the travel stop is configured to limit a maximum extension of the linear actuator.

According to a ninth aspect of the present disclosure, a method of operating a brake system on a patient support apparatus includes extending a linear actuator to rotate a clutch about an axis and cause a shaft assembly to move a caster brake of a caster to a braked position and block rotation of a wheel of the caster, disengaging a first clutch plate from a second clutch plate of the clutch assembly to operably decouple the shaft assembly from the linear actuator, and moving the shaft assembly automatically in response to disengaging the first clutch plate from the second clutch plate to cause the shaft assembly to unbrake the caster brake.

In some embodiments of the method of the ninth aspect, the method may include retracting the linear actuator to rotate the first clutch plate. The method may include moving the second clutch plate into engagement with the first clutch plate to couple the first clutch plate with the second clutch plate after moving the shaft assembly automatically.

In some embodiments of the method of the ninth aspect, disengaging the first clutch plate from the second clutch plate may include moving a plunger operably coupled with the second clutch plate from a first position to a second position. In some embodiments of the patient support apparatus of the ninth aspect, the method may include moving the plunger from the second position toward the first position without engaging the second clutch plate with the first clutch plate before retracting the linear actuator.

In some embodiments of the method of the ninth aspect, the method may include engaging the linear actuator with a travel stop. The travel stop blocks the linear actuator from extending to a fully extended position.

According to a tenth aspect of the present disclosure, a method of synchronizing caster brakes of a patient support apparatus includes moving a first caster brake link to a first predetermined position to cause a first caster brake to be unlocked, moving a second caster brake link relative to the first caster brake link to a second predetermined position to cause a second caster brake to be unlocked, applying a first force to the first caster brake link and the second caster brake link to urge the first caster brake link away from the second caster brake link while the first caster brake and the second caster brake remain unlocked, and fixing the first caster brake link with the second caster brake link for movement with the second caster brake link while the first force is applied to the first caster brake link.

In some embodiments of the method of the tenth aspect, the second caster brake link may include a shaft and a cam coupled with the shaft. Applying the first force to the first caster brake link and the second caster brake link may include engaging the first caster linkage with the cam so that the cam urges the first caster brake link away from the shaft.

In some embodiments of the method of the tenth aspect, the cam may include a washer and a fastener. The washer may have an eccentric profile that engages the first caster brake link. The fastener may couple the washer with the shaft.

In some embodiments of the method of the tenth aspect, one of the first caster brake link and the second caster brake link may be formed to define an elongated slot. Fixing the first caster brake link with the second caster brake link may include clamping the first caster brake link to the second caster brake link with a fastener that extends through the elongated slot and the other of the first caster brake link and the second caster brake link.

In some embodiments of the method of the tenth aspect, the method may include temporarily restricting movement of the first caster brake link away from the first predetermined position after moving the first caster brake link to the first predetermined position and before applying the first force to the first caster brake link and the second caster brake link. In some embodiments of the patient support apparatus of the tenth aspect, the method may include unrestricting the movement of the first caster brake link away from the first predetermined position after coupling the first caster brake link with the second caster brake link.

In some embodiments of the method of the tenth aspect, the first caster brake link may include a first shaft and a fist pivot connector pivotably coupled with the first shaft and coupled with the first caster brake. The second caster brake link may include a second shaft and a second pivot connector pivotably coupled with the second shaft and coupled with the second caster brake.

In some embodiments of the method of the tenth aspect, the method may include pivotably coupling an actuator to an actuator mount and operably coupling the actuator with the first caster brake link. In some embodiments of the patient support apparatus of the tenth aspect, the method may include applying a second force to the actuator mount to urge the actuator mount away from one of the first caster brake and the second caster brake and toward the other of the first caster brake and the second caster brake before applying the first force to the first caster brake link and the second caster brake link.

In some embodiments of the method of the tenth aspect, the method may include fixing the actuator mount with a base of the patient support apparatus. Fixing the actuator mount with the base may be performed before applying the first force to the first caster brake link and the second caster brake link.

According to an eleventh aspect of the present disclosure, a patient support apparatus includes a first caster and a second caster, a brake drive, and a linkage. The first caster includes a first wheel configured to rotate about a first wheel axis and a first braking element movable from an unbraked position allowing rotation of the first wheel about the first wheel axis to a braked position blocking the first wheel from rotation relative to the first wheel axis. The second caster includes a second wheel configured to rotate about a second wheel axis and a second braking element movable from an unbraked position allowing rotation of the second wheel about the second wheel axis to a braked position blocking the second wheel from rotation relative to the second wheel axis. The brake drive includes a mount and a linear actuator coupled to the mount. The linear actuator is movable between a retracted position and an extended position relative to the mount. The linkage is configured to transfer motion of the linear actuator to the first braking element and the second braking element to move the first braking element and the second braking element between the unbraked position and the braked position.

The linkage includes a shaft assembly having a first caster brake link coupled with the first braking element, a second caster brake link coupled with the second braking element and operably coupled with the linear actuator, and a cam coupled with one of the first caster brake link and the second caster brake link. The first caster brake link is movable selectively relative to the second caster brake link. The cam is configured to engage the other of the first caster brake link and the second caster brake link to urge selectively the first caster brake link away from the second caster brake link.

In some embodiments of the patient support apparatus of the eleventh aspect, the cam may include a washer and a fastener. The washer may have an eccentric profile that engages the other of the first caster brake link and the second caster brake link. The fastener may couple the washer with the one of the first caster brake link and the second caster brake link.

In some embodiments of the patient support apparatus of the eleventh aspect, one of the first caster brake link and the second caster brake link may be formed to define an elongated slot. The shaft assembly may include a fastener that extends through the elongated slot and into the other one of the first caster brake link and the second caster brake link to couple the first caster brake link with the second caster brake link.

In some embodiments of the patient support apparatus of the eleventh aspect, the first caster brake link may include a first shaft and a first fork pivotably coupled with the first braking element. The second caster brake link may include a second shaft and a second fork pivotably coupled with the second braking element. In some embodiments of the patient support apparatus of the eleventh aspect, the first caster brake link and the second caster brake link may be biased toward moving the braking elements toward the unbraked position.

In some embodiments of the patient support apparatus of the first, second, third, fourth, fifth, sixth, and seventh, aspects, the patient support apparatus further includes a remote pendant removably coupled with one of the first column and one of the second column and a panel fixed to one of the first lift column and the second lift column. Each of the remote pendant and the panel have a plurality of buttons thereon. A height of the first lift column and the height of the second lift column are adjustable to locate the axis in a horizontal orientation above a floor underlying the support base by a vertical distance. The buttons of the remote pendant and the buttons of the panel are each configured to adjust the height of the first lift column, the height of the second lift column, and rotation of the patient support about this axis.

In some embodiments of the methods of the ninth aspects, the methods further comprise adjusting a position of one of the first lift column, the second lift column, and the patient support in response to receiving a first input from a remote pendant and adjusting the position of one of the first lift column, the second lift column, and the patient support in response to receiving a second input from a panel fixed with one of the first column and the second column.

According to a twelfth aspect of the present disclosure, a patient support apparatus includes a support base, a first lift column and a second lift column spaced apart from the first lift column, a patient support, and a user interface. The first lift column and the second lift column are supported by the support base. The patient support extends between and couples with the first lift column and the second lift column. The patient support is rotatable about an axis that extends between the first lift column and the second lift column. The user interface includes a remote pendant including a plurality of buttons and a panel fixed with one of the first column and the second column and including a plurality buttons. The plurality of buttons of the remote pendant and the plurality of buttons of the panel are each configured to adjust one or more of a height of the first column, a height of the second column, and a rotation of the patient support about the axis.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1A is a perspective view of a patient support apparatus including a foundation frame and a patient support top supported on the foundation frame to rotate about an axis;

FIG. 1B is a diagrammatic view of the patient support apparatus of FIG. 1A suggesting the columns of the patient support apparatus can be raised and lowered individually to raise, lower, and tilt the patient support top;

FIG. 1C is a diagrammatic view of the patient support top of the patient support apparatus of FIG. 1A suggesting that the patient support top is rotatable about an axis between a plurality of positions;

FIG. 2 is a diagrammatic view of the patient support apparatus of FIG. 1A;

FIG. 3 is an elevation view of a remote pendant included with the patient support apparatus of FIG. 1A and configured to control the operation of the patient support apparatus;

FIG. 4 is a top view of a panel located on a column of the foundation frame of the patient support apparatus of FIG. 1A and configured to provide an additional interface to control the operation of the patient support apparatus;

FIG. 5 is a section view of one of the columns of the foundation frame of the patient support apparatus to reveal a rotation system and rotation indicator for providing information indicative of a rotational angle of the support top to a user of the patient support apparatus;

FIG. 6 is a perspective view of the rotation indicator of FIG. 5, the rotation indicator including a marker configured to slide in a guide track and visible through a window of the panel on top of the column of the patient support apparatus;

FIG. 7 is a perspective view of one of the columns of the patient support apparatus having the panel on a top face of the column and an accessory rail coupled to a side of the column with the remote pendant supported thereon;

FIG. 8 is an enlarged view of the side of the column and the accessory rail of FIG. 7;

FIG. 9A is a perspective view of the accessory rail of FIG. 8 having a first rail body with elongated slots formed therein and a second rail body that extends perpendicular away from an end of the first rail body;

FIG. 9B is a front elevation view of the accessory rail of FIG. 9A;

FIG. 9C is a top plan view of the accessory rail of FIG. 9A;

FIG. 9D is a side elevation view of the accessory rail of FIG. 9A;

FIG. 9E is a rear elevation view of the accessory rail of FIG. 9A;

FIG. 10 is a front elevation view of the patient support apparatus of FIG. 1A, the foundation frame of the patient support apparatus including the column and a support base underlying the column and having a light system for displaying a light indicative of a brake status of the casters of the support base;

FIG. 11 is a perspective view of a light emitter included in the light system revealed by removing a cover of the support base; the light emitter configured to direct the light through a window coupled to an underside of the support base and onto a floor underlying the patient support apparatus;

FIG. 12 is a section view of a portion of the support base to reveal the light emitter directing the light through the window and onto the floor to indicate a brake status of the casters;

FIG. 13 is a bottom perspective view of the window coupled with the cover of the support base and configured to allow the light from the light emitter to pass therethrough;

FIG. 14 is another perspective of the window of FIG. 13;

FIG. 15 is a perspective of a lock unit and an extension having telescoping tubes that allow bases of the support base to move relative to each other between a collapsed and deployed position, the lock unit configured to selectively block relative movement of the tubes to maintain the collapsed or deployed positions;

FIG. 16 is a side elevation of one of the tubes of the extension, the tube having a tube body and a band cut from the tube body and coupled with the lock unit to selectively apply a compression force to the other tube of the extension;

FIG. 17 is a section view of the lock unit to reveal a first mount plate, second mount plate, pin, and cam body of the lock unit;

FIG. 18 is a perspective and section view of the lock unit and extension to reveal the lock unit is coupled with the band of the tube to control movement of the band to selectively tighten and loosen the band for the compressive force applied to the inner tube of the extension;

FIG. 19 is a side elevation view of a portion of the patient support apparatus in a lowest low position;

FIG. 20 is a side elevation view of the portion of the patient support apparatus similar to FIG. 19 with the patient support apparatus in a highest high position;

FIG. 21 is an elevation view of the support base of the patient support apparatus with the cover removed to reveal a brake system of the patient support apparatus;

FIG. 22 is a top elevation view of the support base of FIG. 21 with the cover removed to reveal the brake system of the patient support apparatus;

FIG. 23A is a side elevation view of the brake system coupled with the base of the patient support apparatus, the brake system being in the locked position and including a brake drive, a clutch assembly, and a manual release assembly;

FIG. 23B is an enlarged view of a portion of the manual release assembly with the brake system moved partway between the braked and unbraked position suggesting the bias members of the manual release assembly dampen movement and reduce hard stops for the brake system;

FIG. 24 is a side elevation view of the brake system of FIG. 23A in the unlocked position;

FIG. 25 is a perspective and exploded view of the clutch assembly included in the brake system of the patient support apparatus, the clutch assembly including, among other things, a clutch shaft, a first clutch plate, and a second clutch plate configured to selectively disengage the first clutch plate;

FIG. 25A is a perspective and exploded view of another embodiment of a clutch assembly configured for use in the brake system of the patient support apparatus;

FIG. 26 is an elevation and exploded view of the clutch assembly of FIG. 25;

FIG. 26A is an elevation and exploded view of the clutch assembly of FIG. 25A;

FIG. 27 is a perspective view of the clutch assembly of the brake system included in the patient support apparatus in an engaged arrangement with the linear actuator extended to cause the clutch assembly to move the shaft assembly and brake the caster brakes on the support base of the patient support apparatus;

FIG. 28 is a perspective view of the clutch assembly of the brake system of FIG. 27 in an engaged arrangement with the linear actuator retracted to cause the clutch assembly to move the shaft assembly and unbrake the caster brakes on the support base of the patient support apparatus;

FIG. 29 is a top view of the manual release assembly and clutch assembly in the support base of the patient support apparatus, the manual release assembly not activated such that the clutch assembly is engaged and configured to transmit movement between the linear actuator and the braking elements of the casters to brake and unbrake the casters;

FIG. 30 is a top view of the manual release assembly and clutch assembly of FIG. 29, a plunger of the manual release assembly having been pulled to activate the manual release assembly and disengaged the clutch assembly to decouple the linear actuator from the braking elements of the casters to allow the manual release assembly to unbrake the casters;

FIG. 31 is a top view of the manual release assembly and clutch assembly similar to FIG. 30, the plunger of the manual release assembly having been released such that bias elements urge the plunger towards its initial inactivated position and urge the clutch assembly toward the engaged arrangement while remaining disengaged so that the clutch assembly is ready for engagement in response to movement of the linear actuator;

FIG. 32 is an enlarged view of the manual release assembly and clutch assembly of FIG. 30 with the manual release assembly activated to cause the clutch assembly to be in the disengaged arrangement;

FIG. 33 is an enlarged view of the manual release assembly and clutch assembly of FIG. 31 with the plunger of the manual release assembly released after the manual release assembly has been activated to cause the clutch assembly to be in the disengaged arrangement, but biased toward the engaged arrangement and ready to be re-engaged in response to retraction of the linear actuator;

FIG. 34 is a section view of the brake system in the support base of the patient support apparatus, the linear actuator being in a fully extended position to cause the clutch assembly to engage a travel stop to prevent overextension of the linear actuator;

FIG. 35 is an enlarged perspective view of the brake assembly with the braking elements of the casters in the unbraked position;

FIG. 36 is a section view through one of the casters of the patient support apparatus, the braking element of the caster including a hex bolt and the brake system including a fork coupled to an end of the hex bolt for moving the hex bolt between the braked and unbraked position and further including a set screw for blocking rotation of the hex bolt during calibration of the brake system;

FIG. 37 is a section view through the linear actuator and mount of the brake drive located in the support base of the patient support apparatus, the mount being coupled with a plate of the support base via fasteners and the brake system including a jack screw for locating the mount relative to the plate during calibration of the brake system; and

FIG. 38 is a section view through the shaft assembly of the brake system located in the support base of the patient support apparatus, the shaft assembly including a first shaft and a second shaft coupled to the first shaft via fasteners in slotted holes to allow limited movement of the shafts for calibration of the brake system.

### DETAILED DESCRIPTION

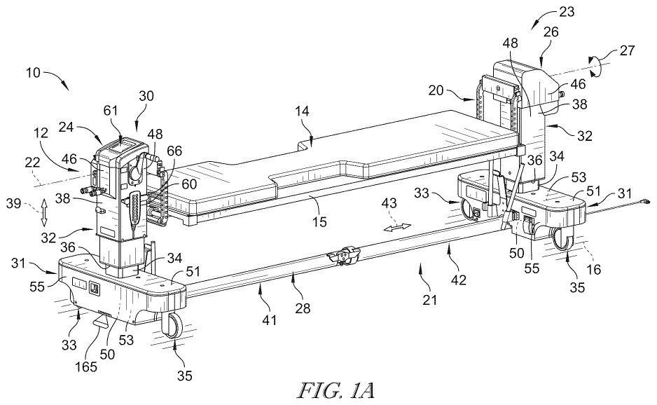

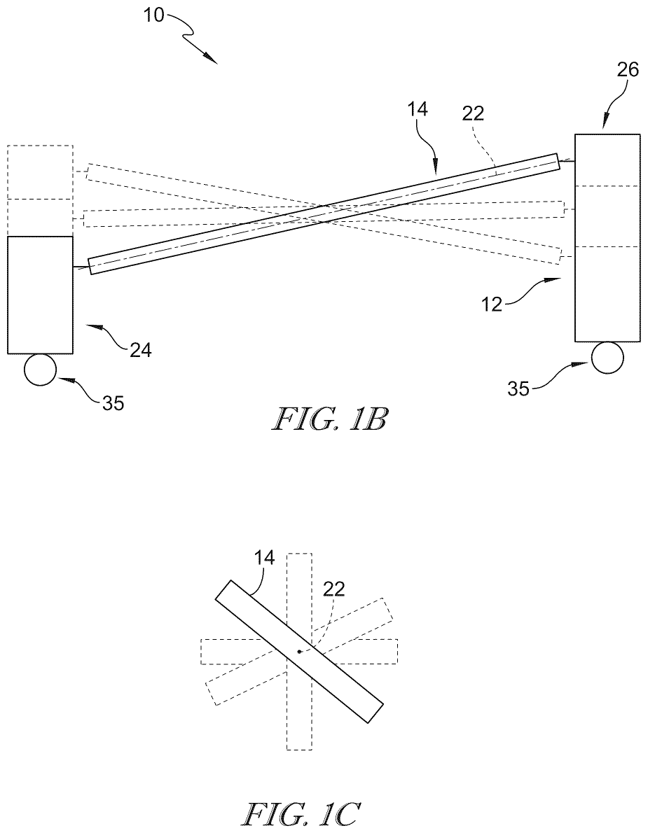

A patient support apparatus 10 for supporting a patient during surgery is shown in FIG. 1A. The patient support apparatus 10 illustratively includes a foundation frame 12 and a patient support 14. The foundation frame 12 rests on a floor 16 and is configured to suspend the patient support 14 in a number of different positions above the floor 16 as suggested in FIGS. 1B and 1C. Thus, a patient undergoing surgery can be moved with the patient support 14 to the number of different positions and orientations depending on the particular surgical operation to be performed on the patient.

The foundation frame 12 includes a support base 21 and a lift system 23 coupled with the support base 21 and configured to raise and lower the patient support 14 relative to the support base 21 as suggested in FIGS. 1A, 1B, 19, and 20. In the illustrative embodiment, the support base 21 and the lift system 23 are provided by a first column 24, a second column 26, an extension 28, and a control system 30 as shown in FIG. 1A. Each column 24, 26 includes a base 31 and a lift column 32 extending up from the respective base 31 as shown in FIG. 1A. The base 31 of the first column 24, the base 31 of the second column 26, and the extension 28 cooperate to provide the support base 21 in the illustrative embodiment. Each base 31 includes a horizontal top plate 51, a vertical lower plate 53 extending down from the horizontal plate 51, and a cover 55 coupled the top plate 51 to house the lower plate 53. The lift column 32 of the first column 24, the lift column 32 of the second column 26, and the control system 30 cooperate to provide the lift system 23 in the illustrative embodiment. The patient support apparatus 10 may support patients with weights up to 650 pounds in the illustrative embodiment. The first column 24 is a head-end column and the second column 26 is a foot-end column in the example patient support apparatus 10 of the present disclosure.

The patient support 14 is coupled to the foundation frame 12 between the columns 24, 26 via yoke brackets 20. The patient support 14 and the yoke brackets 20 are configured to rotate relative to the foundation frame 12 about a pivot axis 22 as suggested by arrow 27 in FIG. 1A and further suggested in the diagrammatic view of the patient support 14 in FIG. 1C. The pivot axis 22 extends parallel to, and is spaced apart from, the length of the patient support 14. The pivot axis 22 extends through the lift column 32 of the first column 24 and the lift column 32 of the second column 32. Thus, a patient can be rotated prior to or during a surgical operation. In some embodiments, the patient support 14 may be rotated 360 degrees about the pivot axis 22 and held in place in the rotated positions. The pivot axis 22 may be tilted relative to the floor 16 by raising and lowering the lift columns 32 of the columns 24, 26 relative to each other as suggested in FIG. 1B. The patient support 14 may be tilted and rotated and held in position in the tilted and rotated arrangement.

Each lift column 32 includes a lower section 34, an intermediate section 36 movable vertically up and down along the lower section 34, and an upper section 38 that is movable vertically up and down along the intermediate section 36 as suggested by arrow 39 to raise and lower each end of the patient support 14. In other words, each lift column 32 includes at least three telescoping segments and an actuator configured to adjust the height of the lift column 32. Thus, a patient can be raised, lowered, or inclined prior to or during a surgical operation at the discretion of a surgeon. In other embodiments, the lift columns 32 include at least two telescoping segments or at least one cantilevered lift arm.

Additionally, each column 24, 26 of the patient support apparatus 10 includes a pair of casters 33, 35 that engage the floor 16 as shown in FIG. 1A. All of the casters 33, 35 are selectively freed to allow the patient support apparatus 10 to roll along the floor 16 to different surgery or storage rooms within a healthcare facility. However, during surgical operations, the patient support apparatus 10 may be held in place relative to the floor 16 by braking the casters 33, 35 to minimize unwanted movement of the patient during the operation.

The extension 28 extends between the columns 24, 26 and includes a first tube 41 and a second tube 42 configured to telescope relative to the first tube 41 as suggested by arrow 43 in FIG. 1A. Telescoping of the tubes 41, 42 allow the columns 24, 26 to be moved between a deployed position and a collapsed position. In the deployed position, the columns 24, 26 are spaced apart to support the patient support 14 therebetween. In the storage position, the columns 24, 26 are moved toward one another reducing the footprint of the foundation frame 12. The first tube 41 is coupled with the base 31 of the head-end column 24 and the second tube 42 is coupled with the base 31 of the foot-end column 26 in the illustrative embodiment. The tubes 41, 42 may have any suitable cross-sectional shape such as, but not limited to, circular, square, semi-circular, I-beam, etc.

The control system 30 is configured to control the motions of the patient support apparatus 10. Specifically, the control system 30 directs rotation of the patient support 14 about the pivot axis 22, movement of the upper section 38, the intermediate section 36, and the lower section 34 of each lift column 32 to raise and lower the ends of the patient support 14, and freedom of the casters 33, 35 to roll along the floor 16.

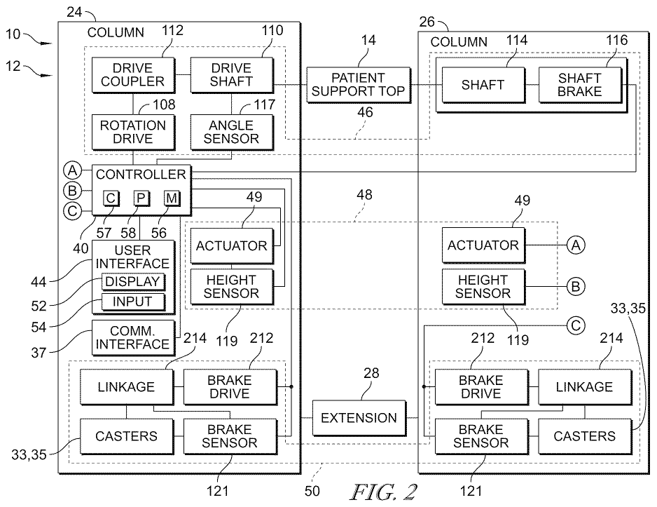

The control system 30 includes a controller 40, a user interface 44, a rotation system 46, a lift drive 48, and a brake system 50 as shown diagrammatically in FIG. 2. The controller 40 is electrically coupled to the user interface 44, the rotation system 46, the lift drive 48, and the brake system 50. The user interface 44 may include a display 52 and a user input 54 configured to allow a surgeon to direct operation of and receive information about the foundation frame systems 46, 48, 50. The rotation system 46 is configured to provide powered and manual rotation of the patient support 14 about the pivot axis 22. The lift drive 48 is configured to provide powered movement of the upper section 38, the intermediate section 36, and the lower section 34 to raise and lower the ends of the patient support 14. The brake system 50 is configured to provide powered braking and unbraking of the casters 33, 35 and to allow manual unbraking of the casters 33, 35 in case of a power failure.

In some embodiments, the control system 30 includes a communications interface 37 connected with the controller 40. The communications interface 37 communicates with a remote computer device via communication infrastructure such as an Ethernet of a healthcare facility in which the patient support apparatus 10 is located and via communication links. The remove computer device is sometimes simply referred to as a “computer” herein. The computer may be part of a surgical computer system, a maintenance computer system, or an electronic medical records (EMR) system, for example. However, it is within the scope of this disclosure for the communication interface 37 of the patient support apparatus 10 to communicate with other computers used in a healthcare facility.

The control system 30 of the patient support apparatus 10 also includes an angle sensor 117, a pair of height sensors 119, and a pair of brake sensors 121 each in communication with the controller 40 as shown in FIG. 2. The angle sensor 117 is included in the rotation system 46 and is configured to detect the angle of the patient support 14 relative to the ground. The height sensors 119 are included in the lift drive 48 and are configured to detect the height of either end of the patient support 14 as a function of the amount of lift applied to the columns 24, 26. The brake sensors 121 are included in the brake system 50 and are configured to detect the position of the linkages 214 to determine the braking status of the casters 33, 35.

The controller 40, shown diagrammatically in FIG. 2, illustratively includes a memory 56 containing instructions, a clock 57, and a processor 58 coupled to the clock 57 and to the memory 56 to execute the instructions stored therein. The memory 56 may be embodied as or otherwise include one or more memory devices or data storage locations including, for example, dynamic random access memory devices (DRAM), synchronous dynamic random access memory devices (SDRAM), double-data rate synchronous dynamic random access memory device (DDR SDRAM), mask read-only memory (ROM) devices, erasable programmable ROM (EPROM), electrically erasable programmable ROM (EEPROM) devices, flash memory devices, and/or other volatile and/or non-volatile memory devices. The processor 58 may be embodied as any type of processor capable of executing the instructions stored in the memory 56. The illustrative processor 58 is a single core processor, but processors having multiple cores may be used in other embodiments.

The user interface 44 of the illustrative embodiment includes a remote pendant 60 and a panel 61 as shown in FIG. 1A. The display 52 and the input 54 of the user interface 4 may be provided by the indicators, lights, and buttons on the remote pendant 60 and the panel 61. The remote pendant 60 is illustratively coupled to the first column 24 by an extendable cord 64 and is removably supported on an accessory side rail 150 situated along a right side 63 of the upper section 38 included in the first column 24. In other embodiments, the remote pendant 60 may be wireless. The panel 61 is illustratively mounted to a top side 65 of the upper section 38 included in the first column 24. The patient support 14 is controllable by either and both of the pendant 60 and panel 61. In other words, the patient support 14 may be adjusted using the pendant 60 and then later during the same operation adjusted using the panel 61 and vice versa.

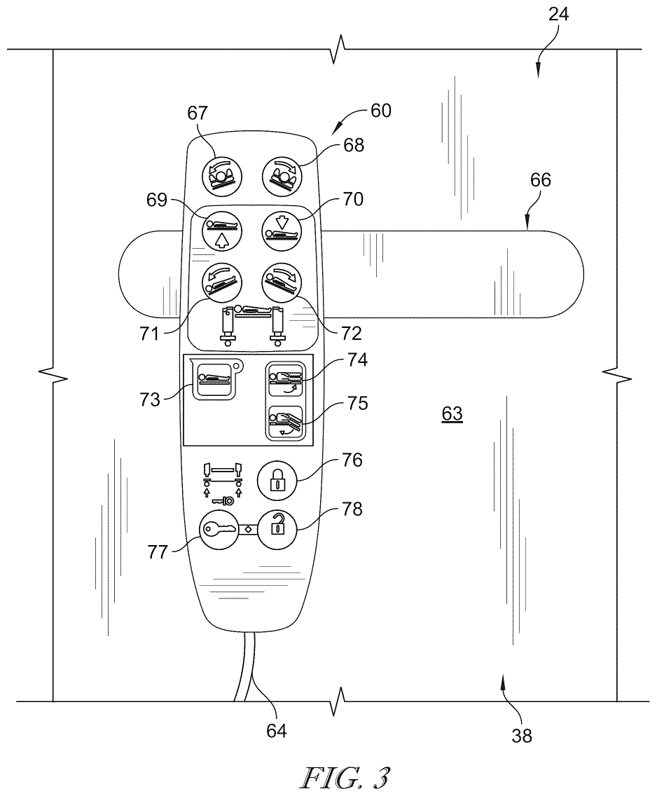

As shown in FIG. 3, the remote pendant 60 includes buttons 67-75. In the illustrative embodiment, buttons 67-75 are electro-mechanical push-button switches arranged adjacent icons suggesting the associated movement type and each button is marked with an indicator to suggest the direction of movement to be activated by pressing the respective button. In other embodiments, the remote pendant 60 may include GUI interfaces such as LCD screens with menus and/or touch sensitive areas to achieve the functions described.

Buttons 67, 68 are configured to activate rotation system 46 to rotate the patient support 14 about the pivot axis 22 as suggested in FIG. 1C. Button 69 is configured to activate actuators 49 of the lift drive 48 to simultaneously raise the upper sections 38 and the intermediate sections 36 of the first column 24 and the second column 26 relative to the lower sections 34 of the first column 24 and the second column 26. Button 70 is configured to activate the actuators 49 of the lift drive 48 to simultaneously lower the upper sections 38 and the intermediate sections 36 of the first column 24 and the second column 26 relative to the lower sections 34 of the first column 24 and the second column 26.

Button 71 on remote pendant 60 of FIG. 3 is configured to tilt the patient support 14 toward or into a Trendelenburg position by activating the actuator 49 included in the lift drive 48 to raise the upper section 38 and the intermediate section 36 of the second column 26 relative to the lower section 34 of the second column 26 and to lower the upper section 38 and the intermediate section 36 of the first column 24 relative to the lower section 34 of the first column to rotate the patient support 14 such that the pivot axis 22 is non-parallel with the floor 16 as suggested in FIG. 1B. Button 72 is configured to tilt the patient support 14 toward or into the reverse Trendelenburg position by activating the actuator 49 included in the lift drive 48 to lower the upper section 38 and the intermediate section 36 of the second column 26 relative to the lower section 34 of the second column 26 and to raise the upper section 38 and the intermediate section 36 of the first column 24 relative to the lower section 34 of the first column to rotate the patient support 14 such that the pivot axis 22 is non-parallel with the floor 16.

Button 73 on remote pendant 60 of FIG. 3 is configured to level the patient support 14 by raising or lowering the upper sections 38 and the intermediate sections 36 of the first and second columns 24, 26 relative to the lower sections 34 of the first and second columns 24, 26 to move the patient support 14 to a level position in a horizontal plane relative to the floor 16. In the illustrative embodiment, the columns 24, 26 are each moved to a height that is about midway between a difference in their present height when button 75 is pressed. As an example, if patient support 14 is in a Trendelenburg position and button 75 is activated, first column 24 raises to about a midpoint height between the current height of the first column 24 and the current height of the second column 26 and the second column 26 lowers to about the same midpoint height. In other embodiments, the first column 24 may move while the second column 26 remains at its current height, the second column 26 may move while the first column 24 remains at its current height, or first and second columns 24, 26 may move in other non-equal amounts relative to each other. In response to the button 73 being activated, the columns 24, 26 move automatically to about an equal or same height. As a result, the axis 22 extends generally horizontally relative to the floor 16.

In the illustrative embodiment, activating button 73 further causes the rotation system 46 to center the patient support 14 automatically. In other words, the rotation system 46 rotates the patient support 14 about the axis 22 to cause the patient support 14 to be in a center (zero angle) position. In such position, the patient is typically face up or face down. Movement of the height of the columns 24, 26 and rotation of the patient support 14 occurs simultaneously in the present embodiment. In some embodiments, the height may change and then the patient support 14 is rotated or the patient support 14 is first rotated and then the height of the columns 24, 26 is changed to level the patient support 14.

Button 74 is configured to raise a patient leg support, of patient supports 14 having pivotable leg support section, from a lowered position to a raised position. Button 74 is configured to lower the patient leg support, of patient supports 14 having the pivotable leg support section, from a raised position to a lowered position.

Button 76 on remote pendant 60 of FIG. 3 is configured to lock the brake system 50 to simultaneously lock each of the casters 33, 35 included in the first column 24 and the second column 26 in response to the button 76 being pressed and, optionally, held down for a predetermined period of time. Activating the button 76 causes the brake lock indicator 82 on the panel 61 to illuminate a first color or otherwise indicate that the brake system 50 is locked. Illustratively, the first color is green. Buttons 77 and 78 are configured to unlock the brake system 50 to simultaneously unlock each of the casters 33, 35 included in the first column 24 and the second column 26 in response to both of the buttons 77, 78 being pressed and, optionally, held down for a predetermined period of time. Activating the buttons 78, 78 causes the brake unlock indicator 82 on the panel 61 to illuminate a second color or otherwise indicate that the brake system 50 is unlocked. Illustratively, the second color is orange.

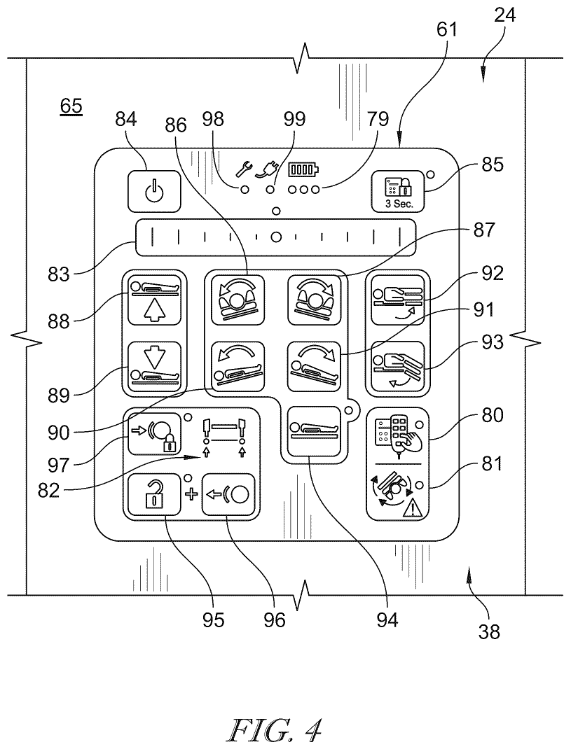

As shown in FIG. 4, the panel 61 includes indicator lights 79-82, 98, 99 showing status information related to the foundation frame 12 and patient support 14 and a rotation indicator 83 indicative of a rotation angle of the patient support 14. The panel 61 further includes buttons 84-97 for controlling the lift drive 48 and brake system 50. In the illustrative embodiment, panel 61 includes a GUI with an LCD for displaying information and/or a touch-screen for displaying information and for receiving user inputs. The panel 61 may include an overlay with the buttons and a polymeric insulation layer located between the overlay and the metallic or other structure of the first column. The polymeric insulation layer may prevent electrical shorting of the buttons with the first column.

A maintenance indicator light 98 is lit when the controller 40 determines that the foundation frame 12 needs service, thereby showing service status. When the maintenance indicator light 98 is lit, all powered functions of the foundation frame 12 may be disabled. A power indicator light 99 is lit when the control system 30 has power received from an external power source (not shown) and a battery power indicator 99 shows battery power status. The brake indicator lights 82 may show a first color or may not be illuminated when the brake system 50 is unbraked so that casters 33, 35 are free to roll along the floor 16, thereby showing brake status and may show a second color or be illuminated in response to the brake system 50 being in the braked position.

A pendant control indicator 80 may be lit when the remote pendant 60 is enabled for control. A flip indicator light 81 is lit when the rotation system 46 is configured to allow manual rotation of the patient support 14 about the pivot axis 22, thereby showing rotation status. A rotation indicator 83 indicates the rotation angle of the rotation system 46, thereby showing rotation angle status of the patient support 14 relative to the foundation frame 12. The rotation indicator 83 is analogue in the illustrative embodiment, but may be a digital indicator in some embodiments.

The power button 84 turns the control system 30 on and off. The panel lock button 85 locks activation of the buttons on the panel 61. The buttons 86 and 87 provide functions similar to buttons 67 and 68 on the remote pendant 60. The buttons 88 and 89 provide functions similar to buttons 69 and 70 on the remote pendant 60. The buttons 90 and 91 provide functions similar to buttons 71 and 72 on the remote pendant 60. The buttons 92 and 93 provide functions similar to buttons 74 and 75 on the remote pendant 60. The button 94 provides a function similar to the button 73 on the remote pendant 60. The button 97 locks the casters 33, 35 similar to button 76 and the buttons 95 and 96 provide caster unlock functions similar to buttons 77 and 78 on the remote pendant 60.

The memory 56 includes instructions that are executable by the processor 58 to perform the functions associated with the buttons 67-77 and buttons 84-97. With respect to the leveling buttons 73, 94, the instructions cause the first lift column 32 and the second lift column 32 to move from a first arrangement in which the first drive shaft 110 of the rotation system 46 is at a first height and the second drive shaft 114 is at a second height different from the first height to a second arrangement in which the first shaft 110 is at a third height and the second shaft 114 is at the third height. The third height is between the first height and the second height in the illustrative embodiment. The third height may be substantially halfway between the first height and the second height. The instructions further cause the first lift column 32 and the second lift column 32 to rotate the patient support 14 about the axis 22 to a predetermined orientation relative to the lift system 23. In the illustrative embodiment, the predetermined orientation is a zero degrees position in which the support top is configured to orient a patient supported thereon in a face up or face down arrangement. Changes in the height and rotational angle of the patient support 14 are performed simultaneously in the illustrative embodiment.

The rotation system 46 for rotating the patient support 14 relative to the foundation frame 12 is housed in the columns 24, 26 of the foundation frame 12 and is coupled to the patient support 14 as shown diagrammatically in FIG. 2. The rotation system 46 is housed in the first lift column 32 and the second lift column 32 of the columns 24, 26. The rotation system 46 is operable in a powered mode to rotate the patient support 14 about the pivot axis 22 in response to a user pressing one of the buttons 67, 68 on the remote pendant 60 or buttons 86, 87 on the panel 61. Thus, a user can rotate a patient without adjusting mechanical locks or manually controlling turning of the patient. Alternatively, in a manual mode, the rotation system 46 allows manual rotation of the patient support 14 about the pivot axis 22 relative to foundation frame 12.

The rotation system 46 includes a rotation drive 108, a drive shaft 110 configured to be operably coupled to the patient support 14, and a drive coupler 112 as shown in FIG. 2. The drive coupler 112 connects the rotation drive 108 to the drive shaft 110 when the rotation system 46 is in the powered mode and disconnects the rotation drive 108 from the drive shaft 110 when the rotation system is in the manual mode. The rotation drive 108, drive shaft 110, and drive coupler 112 are included in the lift column 32 of the first column 24. The rotation system 46 further includes a shaft 114 included in the lift column 32 of the second column 26 and configured to operably couple with the patient support 14 and a shaft brake 116 illustratively coupled with the shaft 114. The patient support apparatus 10 includes yoke brackets 20, sometimes called “H” brackets, that are coupled between the rotation system 46 of the foundation frame 12 and the patient support 14 as shown in FIG. 1A. The yoke brackets 20 couple the shafts 110, 114 to the patient support 14.

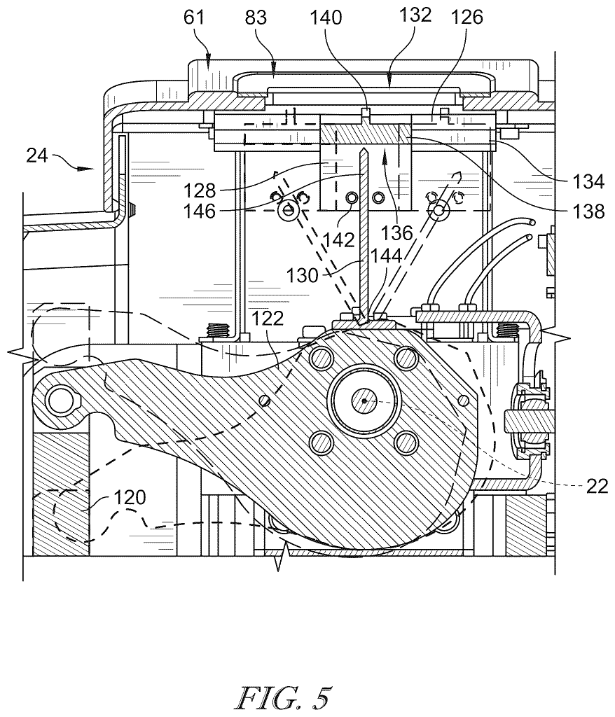

The rotation drive 108 includes a linear actuator 120 and a rotation arm 122 coupled to the linear actuator 120 as shown in FIG. 5. The linear actuator 120 extends and retracts in response to a user operating buttons 67, 68 of remote pendant 60 and buttons 86, 87 of panel 61. The linear actuator 120 further retracts or extends in response to operation of the buttons 73, 94 to level the patient.

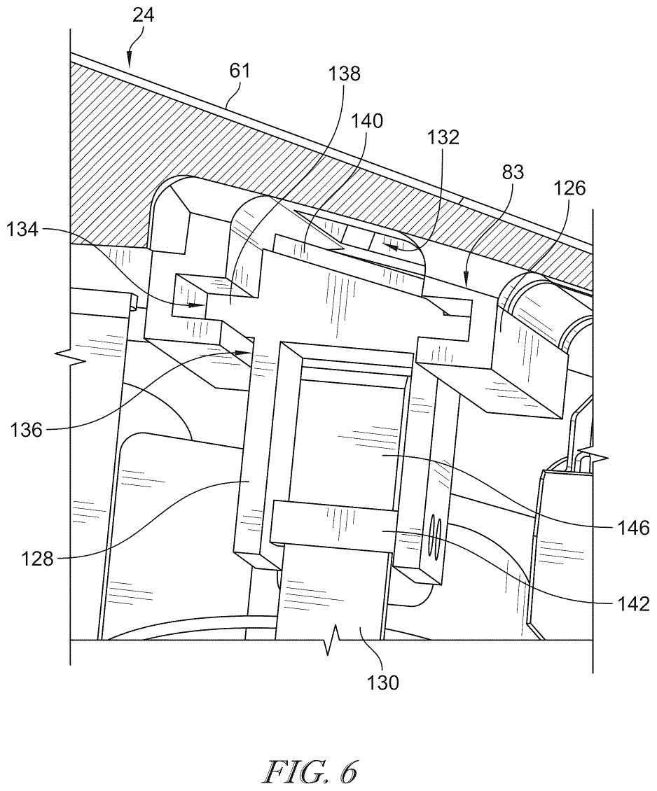

The rotation indicator 83 is operably coupled with the rotation arm 122 as shown in FIG. 5. The rotation indicator 83 is configured to display information indicative of an angle of rotation of the patient support 14 about the axis 22. The rotation indicator 83 is coupled to a top face of the lift column 32 of the first column 24. The rotation indicator 83 includes a gauge track 126, a pointer 128, and a slide lever 130 as shown in FIGS. 5 and 6. The gauge track 126 defines a path for the pointer 128 to move along. The pointer 128 is visible by the user through an opening 132 in the panel 61 and moves along the path of the gauge track 126 to indicate the rotation angle of the patient support 14 in response to rotation of the patient support 14 about the axis 22. The panel 61 and/or gauge track 126 include markings or tick marks at predetermined locations such that the relative position of the pointer 128 and the markings provides the indication of rotation angle to the user. Such tickings may be labeled with numbers or other indicia that is indicative of the angle of rotation. The slide lever 130 is operably coupled with the rotation arm 122 and rotated with the rotation arm 122.

The gauge track 126 is formed to define the pointer display opening 132 that exposes the pointer 128 to a user and a guide slot 134. The gauge track 126 is fixed with the lift column 32 and the guide slot 134 extends laterally along a linear path. The illustrative guide slot 134 extends into a proximal and a distal end of the gauge track 126. The gauge track 126 further includes an opening 136 that opens into the guide slot 134 to receive the pointer 128. The pointer 128 includes a pointer body 138 that extends into the guide slot 134 to slide along the guide slot 134 and a marker 140 coupled with the pointer body 138 and arranged such that at least a portion of the marker 140 is visible to the user through the opening 132. The pointer 128 further defines a cavity that receives a portion of the slide lever 130 therein. In the illustrative embodiment, a first and a second pin 142 extend into the cavity to cooperate with the slide lever 130. In other embodiments, the slide lever 130 may act on other portions, features, or walls of the pointer 128.

The slide lever 130 is configured to rotate about the axis 22 and engage with the pointer 128 to cause the pointer 128 to move in the guide slot 134 in response to rotation of the patient support 14 to indicate to the user the angle of rotation of the patient support 14 about the axis. The slide lever 130 extends in a cantilever relationship from a first end 144 coupled with the rotation arm 122 of the first lift column 32 to a terminal second end 146 that extends into the cavity of the pointer body 138. The second end 146 engages the pins 142 or other structure of the marker 140 to urge the marker 140 to slide along the path of the guide slot 134.

During use of the patient support apparatus 10, the actuator 120 rotates the rotation arm 122 to rotate the patient support 14 in response to an input from the user or the rotation arm 122 may be manually manipulated. Rotation of the rotation arm 122 causes the slide lever 130 to rotate about the axis 22 and urge the marker 140 to slide in the guide slot 134 of the gauge track 126. The rotational movement of the slide lever 130 is transmitted to the marker 140 as linear motion as the slide lever 130 rotates along an arc and the portion of the marker 140 engaging the slide lever 130 slides relative to the slide lever 130. The tick marks on the panel 61 or gauge track 126 are calibrated to correspond with the angle of the patient support 14 based on the linear movement of the marker 140 along the guide slot 134. For example, the spacing between neighboring tick marks may become smaller as the tick marks move laterally away from a center (zero position) of the guide slot 134. In other words, the spacing between tick marks for 30 and 45 degrees of rotation may be smaller than a spacing between tick marks for 0 and 15 degrees of rotation. Rotation of the rotation arm 122 in the opposite direction causes the slide lever 130 to urge the marker 140 in the opposite direction along the guide slot 134 toward the center of the slot 134 and, if further rotation occurs, laterally beyond the center of the guide slot 134.

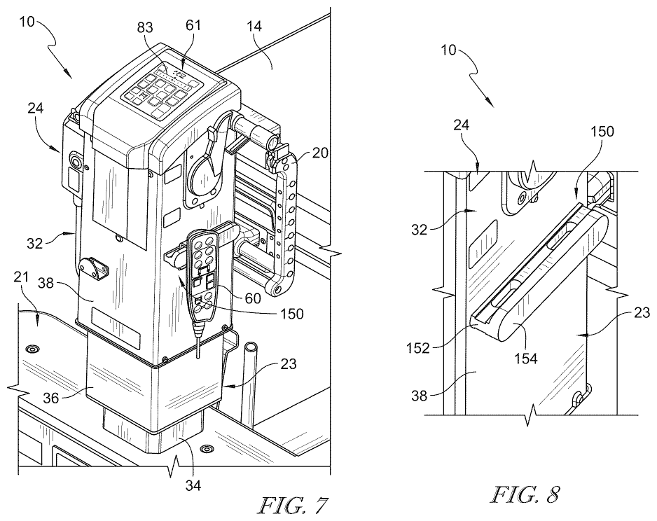

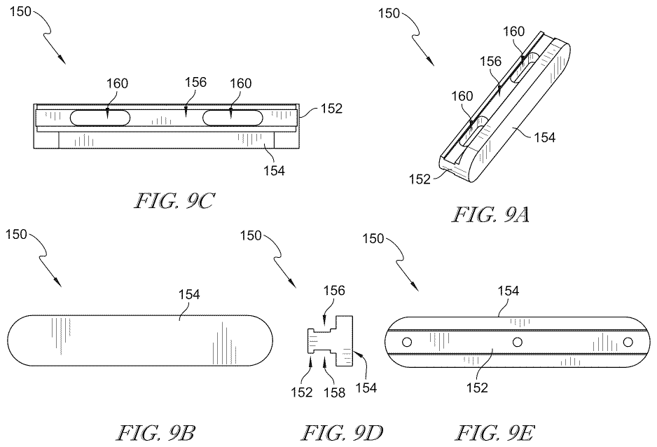

The lift column 32 of the first column 24 further includes an accessory rail 150 coupled to a sidewall of the lift column 32 as shown in FIGS. 7-9D. The accessory rail 150 is fixed with the lift column 32 and configured to support an accessory component on the lift column 32 in removably coupled relationship. The accessory component may include the remote pendant 60, a surgery tool, and support structures such as tables, trays, additional rails, etc. The accessory rail 150 includes a first rail body 152 extending laterally away from the first lift column 32 and a second rail body 154 that extends anteriorly and posteriorly away from an end of the first rail body 152 as shown in FIGS. 9A-9D. The first rail body 152 is formed to define a first channel 156 extending posteriorly partway into the first rail body 152 and a second channel 158 extending anteriorly partway into the first rail body 152.

The first rail body 152 is formed to include one or more elongated slots 160 that extend through the first rail body 152 and opens into the first channel 156 and the second channel 158. In the illustrative embodiment, the one or more elongated slots 160 include two slots that are spaced apart from one another in a lateral direction. The elongated slots 160 extend into the first rail body 152 in the posterior-anterior direction entirely through the first rail body 152. The accessory rail 150 is coupled to a side of the telescoping upper section 38 of the lift column 32.

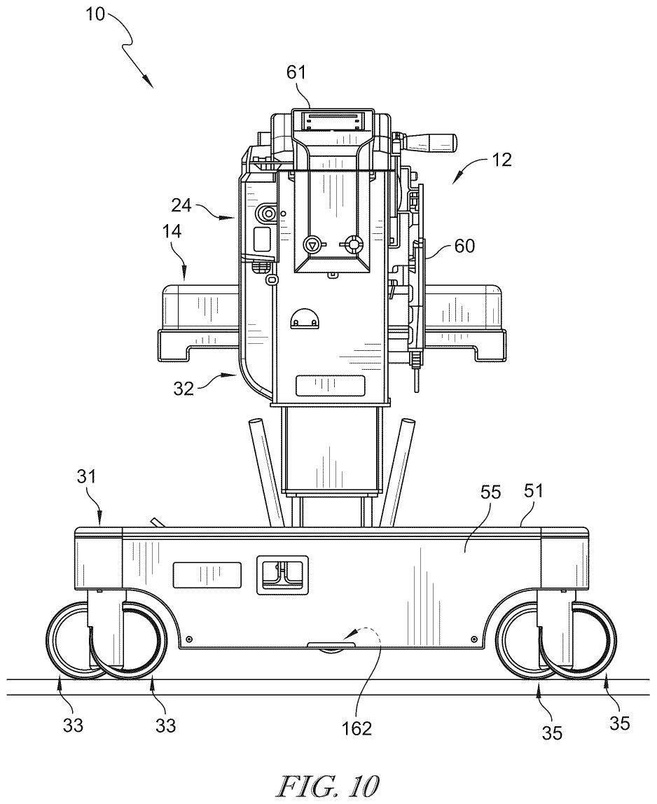

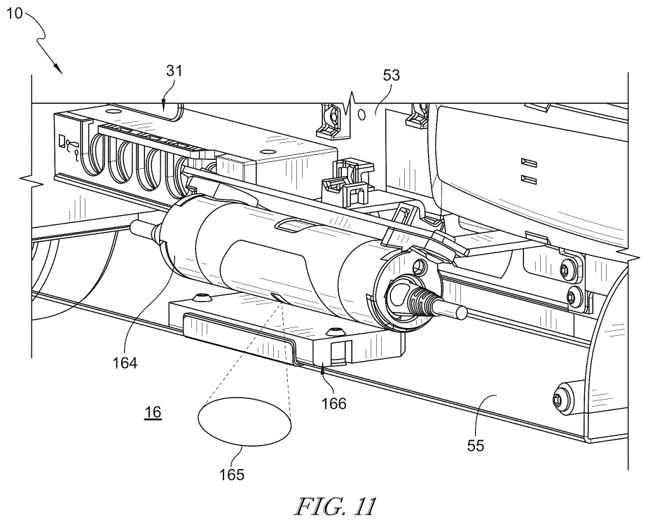

The patient support apparatus 10 includes a caster brake indicator system 162 configured to illuminate a light indicative of brake status of the casters 33, 35 as suggested in FIGS. 10-14. As described in further detail below, each caster 33, 35 includes a stem 312, a wheel 316, and a braking element 318. The wheel 316 is coupled to the stem 312 to rotate about a wheel axis relative to the stem 312. The braking element 318 is movable from an unbraked position allowing rotation of the wheel 316 relative to the stem 312 to a braked position blocking the wheel 316 from rotation relative to the stem 312. The caster brake indicator system 162 illuminates one or more lights to indicate the braking elements 318 being in the braked position and/or the unbraked position. The brake system 50 is configured to move the braking elements 318 between the braked and unbraked positions and illustratively includes a linear actuator 218 that drives movement of the braking elements 318.

The caster brake indicator system 162 includes a light emitter 164 and a window 166 as shown in FIG. 11. The light emitter 164 is coupled with the vertical plate 53 and electrically connected with the controller 40 and to the brake system 50 via the controller 40. The light emitter 164 includes the at least one light and is configured to energize and illuminate the light to provide an indicia 165 indicative of the status of the caster brakes being braked or unbraked. The at least one light may include a single lamp that provides one or mor different colors or multiple lamps that provide different colors. The window 166 is coupled with the cover 55 and is translucent or transparent to allow the light emitter 164 to illuminate the light through the window 166. In the illustrative embodiment, the light emitter 164 and the window 166 are arranged to illuminate the floor underlying the patient support apparatus 10 with the light to provide indicia 165 indicative of a status of the caster brakes.

The light emitter 164 is configured to emit a first color in response to the braking elements 318 being in the braked position and a second color in response to the braking elements 318 being the unbraked position. The second color is a different color from the first color. The first color may be yellow, amber, orange, or red and the second color may be green as illustrative examples. In some embodiments, the light emitter 164 is configured to illuminate the light to provide the indicia 165 in response to the braking elements 318 being only in the unbraked position or only in the braked position. In some embodiments, the light emitter 164 or window 166 include an indicia provided thereon such that the light illuminates the floor with the indicia. For example, the indicia 165 may be colored as described above and/or may also show a symbol, word, shape, etc. in the illuminated area.

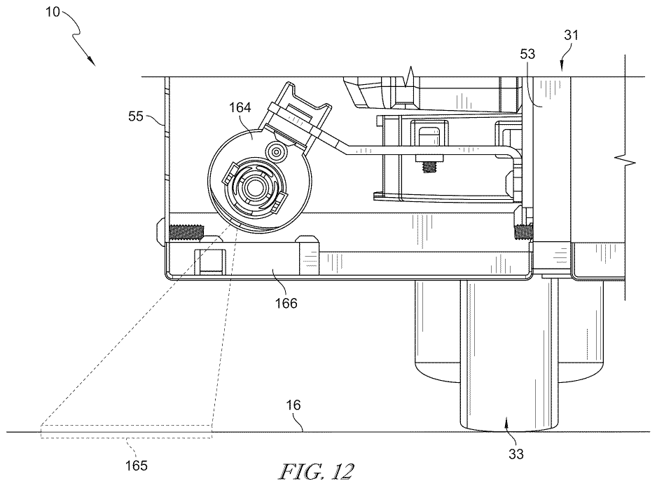

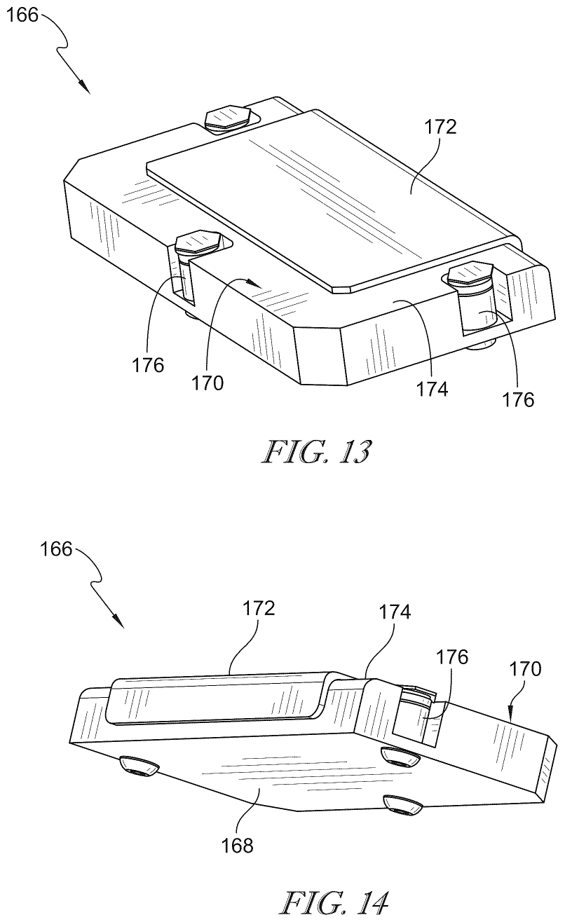

The window 166 is positioned at a proximal most end of the base 31 and about midway between the casters 33, 35 as shown in FIG. 10. As a result, the light is emitted at and illuminates an area of the floor 16 in front of the first column 24 as suggested in FIGS. 1A and 12. The window 166 has an upper surface 168 and a lower surface 170 as shown in FIGS. 13 and 14. The lower surface 170 includes a raised segment 172 that extends through an opening in the cover 55 and a mount surface 174 that extends along the raised segment 172. The window 166 includes fasteners 176 that extend trough the lower surface 170 and the upper surface 168 to couple the window 166 with the cover 55. The window 166 is formed to include recesses 178 that extend into the lower surface 170 toward the upper surface 168 and receive a majority of the fasteners 176 therein.

The extension 28 extends between and interconnects the first base 31 of the first column 24 and the second base 31 of the second column 26 as shown in FIG. 1A. The extension 28 is configured to allow the support base 21 to move between a deployed position in which the first base 31 is spaced apart from the second base 31 by a first distance as shown in FIG. 1 and a collapsed position in which the first base 31 is spaced apart from the second base 31 by a second distance that is less than the first distance.

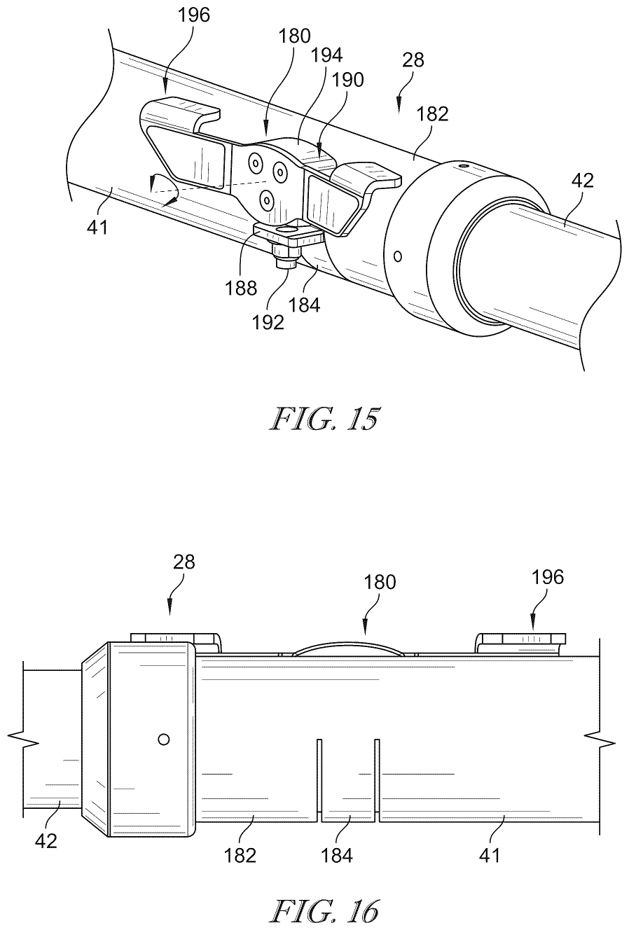

The extension 28 includes a lock unit 180 configured to allow and block the extension 28 from moving between the deployed and collapsed positions as suggested in FIGS. 15-18. The lock unit 180 is configured to move between a locked arrangement and an unlocked arrangement. In the locked arrangement, relative movement between the first tube 41 and the second tube 42 of the extension 28 is blocked. In the unlocked arrangement, relative movement between the first tube 41 and the second tube 42 is allowed to move the support base 21 between the collapsed position and the deployed position.

The lock unit 180 is coupled with the first tube 41 as shown in FIG. 16. The first tube 41 includes a tube body 182 and a band 184 coupled with the tube body 182. The tube body 182 is cut to form the band 184 such that the tube body 182 and the band 184 are integrally formed. The lock unit 180 is configured to move selectively the band 184 relative to the tube body 182 of the first tube 41 to apply a clamping force to the second tube 42 and block relative movement between the first tube 41 and the second tube 42.

The lock unit 180 includes a first mount plate 186, a second mount plate 188, and a pedal assembly 190 as shown in FIGS. 15, 17, and 18. The first mount plate 186 is fixedly coupled with the tube body 182 of the first tube 41. The second mount plate 188 is coupled with a free end of the band 184 to move the band 184 relative to the tube body 182. The pedal assembly 190 is coupled with the first mount plate 186 and the second mount plate 188 to move, selectively, the second mount plate 188 relative to the first mount plate 186 thereby causing the band 184 to apply the clamping force to the second tube 42 and blocking movement between the first tube 41 and the second tube 42.

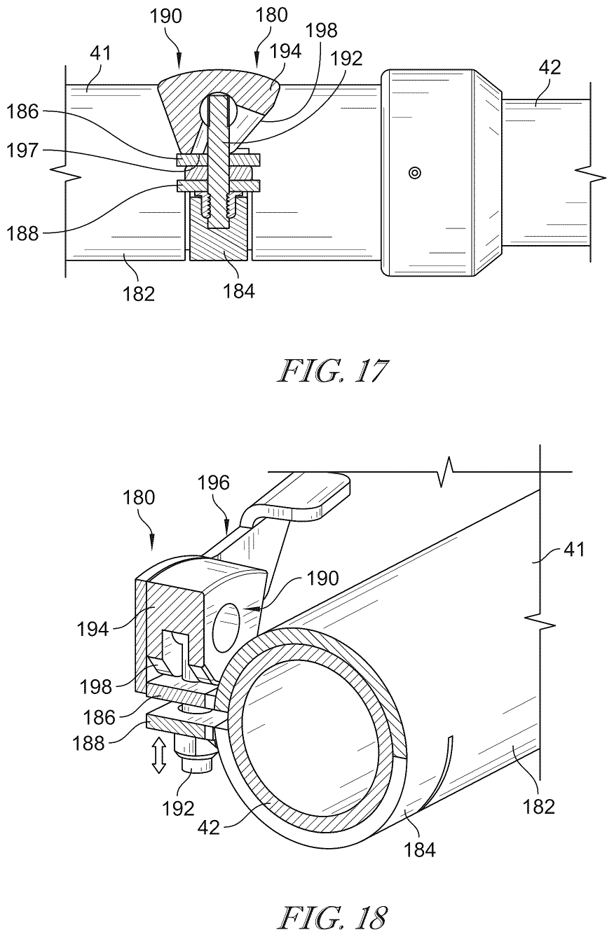

The pedal assembly 190 includes a pin 192 coupled with the second mount plate 188, a cam body 194 coupled with the pin and configured to move the pin 192, and a foot lever 196 configured to control movement of the cam body 194 as shown in FIGS. 17 and 18. The pin 192 extends through the second mount plate 188 and is coupled with the second mount plate 188 via a nut, threading, welding, etc. The pin 192 extends through the first mount plate 186 and is free to move relative to the first mount plate 186. As such, movement of the pin 192 moves the second mount plate 188 and the band 184 relative to the first mount plate 186 and the tube body 182. The cam body 194 is pivotably coupled with the pin 192 and engaged with the first mount plate 186. The cam body 194 has a cam surface having a first portion 197 arranged to engage the first mount plate 186 and move the pin 192 to urge the second mount plate 188 toward the first mount plate 186 and apply the compressive force to the second tube 42 to lock the extension 28.

The cam surface has a second portion 198 arranged to engage the first mount plate 186 and allow the pin 192 to move such that the second mount plate 188 moves away from the first mount plate 186 and reduces or eliminates the compressive force of the second tube 42 to unlock and allow movement of the extension 28. The foot lever 196 is coupled with the cam body 194 and configured to rotate the cam body 194 relative to the first mount plate 186. The foot lever 196 has a first pedal and a second pedal spaced apart from the first pedal. The user may step on each of the first pedal and the second pedal to rotate the foot lever 196 and the cam body 194 about an axis to change the portions 197, 197 of the cam surface engaged with the first mount plate 186 to thereby lock and unlock the lock unit 180.

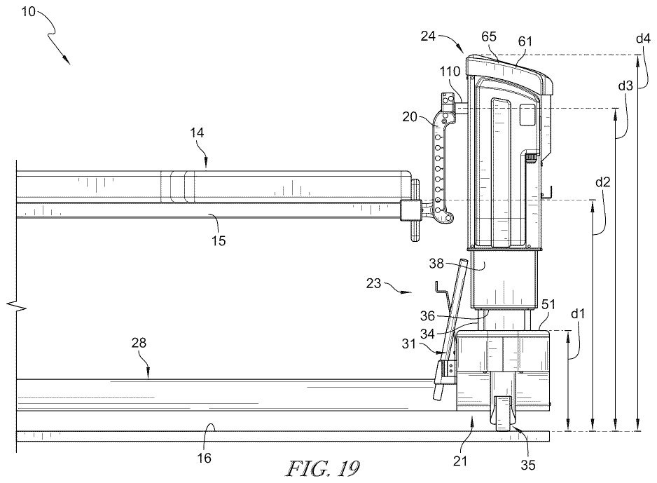

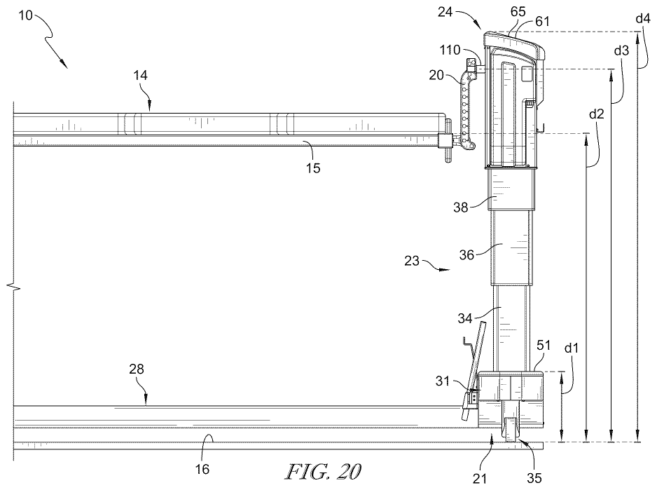

The patient support apparatus 10 is height adjustable to raise and lower the patient support 14 as suggested in FIGS. 19 and 20. The telescoping lift columns 32 of the first column 24 and the second column 26 are configured to minimize a height of the patient support 14 when the patient support apparatus 10 is in the lowermost position as compared to conventional patient support apparatuses while providing desired height in the raised position. In the illustrative embodiment, the minimized lowest low position and desired raised position height are provided by the three or more telescoping sections 34, 36, 38 of the lift columns 32.

A base height d1 is defined as a vertical distance between the floor 16 and an upper surface of the horizontal top plate 51 included in the base 31 of each column 24, 26 as suggested in FIGS. 19 and 20. The base height d1 remains the same in the lower position and the raised position of the lift columns 32 of the lift system 23. In the illustrative embodiment, the base height d1 is equal to about 246 millimeters or less. In some embodiments, the base height d1 is equal to about 250 millimeters or less. In some embodiments, the base height d1 is equal to about 200 millimeters or less. In some embodiments, the base height d1 is equal to about 300 millimeters or less.

A table height d2 is defined as a vertical distance between the floor 16 and an upper surface of a frame 15 included in the patient support 14 with the patient support 14 coupled with a lowest connection point of the yoke bracket 20 as suggested in FIGS. 19 and 20. In the lower position of the lift columns 32 of the lift system 23, the table height d2 is equal to about 560 millimeters or less in the illustrative embodiment. In some embodiments, the table height d2 is equal to about 600 millimeters or less when the lift columns 32 are in the lower position. In some embodiments, the table height d2 is equal to about 580 millimeters or less when the lift columns 32 are in the lower position. In some embodiments, the table height d2 is equal to about 550 millimeters or less when the lift columns 32 are in the lower position.

In the raised position of the lift columns 32, the table height d2 is equal to about 1060 millimeters or more in the illustrative embodiment. In some embodiments, the table height d2 is equal to about 900 millimeters or more when the lift columns 32 are in the raised position. In some embodiments, the table height d2 is equal to about 950 millimeters or more when the lift columns 32 are in the raised position. In some embodiments, the table height d2 is equal to about 1000 millimeters or more when the lift columns 32 are in the raised position. In some embodiments, the table height d2 is equal to about 1050 millimeters or more when the lift columns 32 are in the raised position. In some embodiments, the table height d2 is equal to about 1100 millimeters or more when the lift columns 32 are in the raised position.

A pivot axis height d3 is defined as a vertical distance between the floor 16 and the pivot axis 22 extending through the shafts 110, 114 of the rotation system 46 as shown in FIGS. 19 and 20. The pivot axis 22 is generally horizontal when the columns 24, 26 are in the lower position and in the raised position. In the lower position of the lift columns 32 of the lift system 23, the pivot axis height d3 is equal to about 790 millimeters or less in the illustrative embodiment. In some embodiments, the pivot axis height d3 is equal to about 800 millimeters or less when the lift columns 32 are in the lower position. In some embodiments, the pivot axis height d3 is equal to about 850 millimeters or less when the lift columns 32 are in the lower position. In some embodiments, the pivot axis height d3 is equal to about 900 millimeters or less when the lift columns 32 are in the lower position. In some embodiments, the pivot axis height d3 is equal to about 750 millimeters or less when the lift columns 32 are in the lower position. In some embodiments, the pivot axis height d3 is equal to about 700 millimeters or less when the lift columns 32 are in the lower position.

In the raised position of the lift columns 32 of the lift system 23, the pivot axis height d3 is equal to about 1290 millimeters or more in the illustrative embodiment. In some embodiments, the pivot axis height d3 is equal to about 1300 millimeters or more when the lift columns 32 are in the raised position. In some embodiments, the pivot axis height d3 is equal to about 1350 millimeters or more when the lift columns 32 are in the raised position. In some embodiments, the pivot axis height d3 is equal to about 1400 millimeters or more when the lift columns 32 are in the raised position. In some embodiments, the pivot axis height d3 is equal to about 1250 millimeters or more when the lift columns 32 are in the raised position. In some embodiments, the pivot axis height d3 is equal to about 1,200 millimeters or more when the lift columns 32 are in the raised position. In some embodiments, the pivot axis height d3 is equal to about 1100 millimeters or more when the lift columns 32 are in the raised position.

A column top height d4 is defined as a vertical distance between the floor 16 and a uppermost point of the top side 65 of the columns 24, 26 as shown in FIGS. 19 and 20. In the lower position of the lift columns 32 of the lift system 23, the column top height d4 is equal to about 790 millimeters or less in the illustrative embodiment. In some embodiments, the column top height d4 is equal to about 920 millimeters or less when the lift columns 32 are in the lower position. In some embodiments, the column top height d4 is equal to about 950 millimeters or less when the lift columns 32 are in the lower position. In some embodiments, the column top height d4 is equal to about 1000 millimeters or less when the lift columns 32 are in the lower position. In some embodiments, the column top height d4 is equal to about 900 millimeters or less when the lift columns 32 are in the lower position.

In the raised position of the lift columns 32 of the lift system 23, the column top height d4 is equal to about 1420 millimeters or more in the illustrative embodiment. In some embodiments, the column top height d4 is equal to about 1450 millimeters or more when the lift columns 32 are in the raised position. In some embodiments, the column top height d4 is equal to about 1500 millimeters or more when the lift columns 32 are in the raised position. In some embodiments, the column top height d4 is equal to about 1400 millimeters or more when the lift columns 32 are in the raised position. In some embodiments, the column top height d4 is equal to about 1350 millimeters or more when the lift columns 32 are in the raised position.

The height of the first lift column 32 and the height of the second lift column 32 of the lift system 23 are adjustable such that a ratio between a highest value of the vertical distance of the pivot axis height d3 corresponding to the lift system 23 being in the raised position and a lowest value of the vertical distance of the pivot axis height d3 corresponding to the lift system 23 being in the lower position is greater than or equal to 1.5:1. In some embodiments, the ratio is greater than or equal to 1.3:1. In some embodiments, the ratio is greater than or equal to 1.6:1. In some embodiments, the ratio is greater than or equal to 2:1. In some embodiments, the ratio is less than or equal to 2:1.

The height of the first lift column 32 and the height of the second lift column 32 of the lift system 23 are adjustable relative to the base 31 such that a ratio between the pivot axis height d3 when the pivot axis 22 is in a horizontal orientation from the floor 16 and the base height d1 is variable between at least 3.5:1 (in the lower position) and 5:1 (in the raised position) as the lift system 23 moves between the lower and raised positions. For example, in the lower position, the ratio may be about 3.5:1 or less and in the raised position, the ratio may be about 5:1 or more.

In some embodiments, the ratio between the pivot axis height d3 and the base height d1 is about 3.2:1 when the lift system is in the lower position. In some embodiments, the ratio between the pivot axis height d3 and the base height d1 is about 2.33:1 when the lift system is in the lower position. In some embodiments, the ratio between the pivot axis height d3 and the base height d1 is about 4:1 when the lift system is in the lower position. In some embodiments, the ratio between the pivot axis height d3 and the base height d1 is about 3.5:1 when the lift system is in the lower position.

In some embodiments, the ratio between the pivot axis height d3 and the and the base height d1 is about 7:1 when the lift system is in the raised position. In some embodiments, the ratio between the pivot axis height d3 and the and the base height d1 is about 3.66:1 when the lift system is in the raised position. In some embodiments, the ratio between the pivot axis height d3 and the and the base height d1 is about 5:1 when the lift system is in the raised position. In some embodiments, the ratio between the pivot axis height d3 and the and the base height d1 is about 4.66:1 when the lift system is in the raised position.

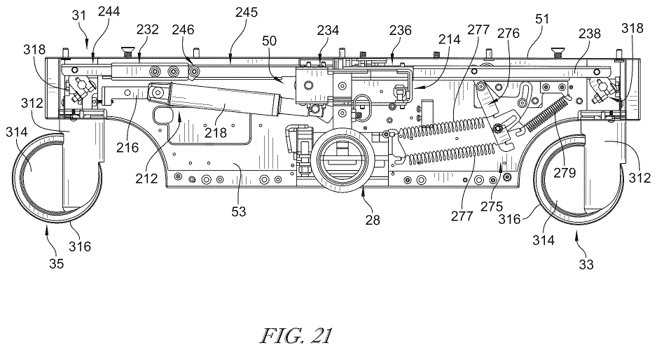

The brake system 50 for holding the patient support apparatus in place on the floor 16 includes a brake drives 212, casters 33, 35, and linkages 214 for each of the first base 31 of the first column 24 and the second base 31 of the second column 26 as shown diagrammatically in FIG. 2 and illustratively in FIGS. 21-38. For ease of description, only components for one base 31 is further described below, the components for the other base are substantially similar to those described below. Thus, the following description is applicable to each of the components included in base 31.

The brake drive 212 is coupled to the controller 40 and is configured to drive the braking system 50 between a braked position and an unbraked position in response to a user input such as buttons 76-78 and 95-97 of the remote pendant 60 and panel 61. The linkage 214 is coupled between the brake drive 212 and the casters 33, 35. The linkage 214 is configured to transfer motion of the brake drive 212 to the braking elements 318 of the casters 33, 35 to move the braking elements 318 between the unbraked position and the braked position. The linkage 214 is further configured to allow a user to manually free the braking system 50 to move the braking elements from the braked position to the unbraked position. The manual unbraking allows a user to move the patient support apparatus 10 in case of a power failure, an emergency, or an equipment failure.

The brake drive 212 includes a mount 216 and a linear actuator 218 as shown in FIGS. 21-23. The mount 216 is coupled to the lower plate 53 included in one of the columns 24, 26 and supports the linear actuator 218. The linear actuator 218 is pivotably coupled to the mount 216 by a bolt and extends and retracts in response to a user input to the remote pendant 60 or panel 61. The linear actuator 218 is further operably coupled with the linkage 214 to drive movement selectively of the linkage 214.

The linear actuator 218 is sized and powered to move the braking elements 318 from the unbraked position to the braked position in less than about 2 seconds and from the braked position to the unbraked position in less than about 2 seconds. The linear actuator 218 may be sized and powered to move the braking elements 318 from the unbraked position to the braked position in less than about 4 seconds and from the braked position to the unbraked position in less than about 4 seconds. The linear actuator 218 may be sized and powered to move the braking elements 318 from the unbraked position to the braked position in less than about 3 seconds and from the braked position to the unbraked position in less than about 3 seconds. The linear actuator 218 may be sized and powered to move the braking elements 318 from the unbraked position to the braked position in less than about 1.75 seconds and from the braked position to the unbraked position in less than about 1.75 seconds.

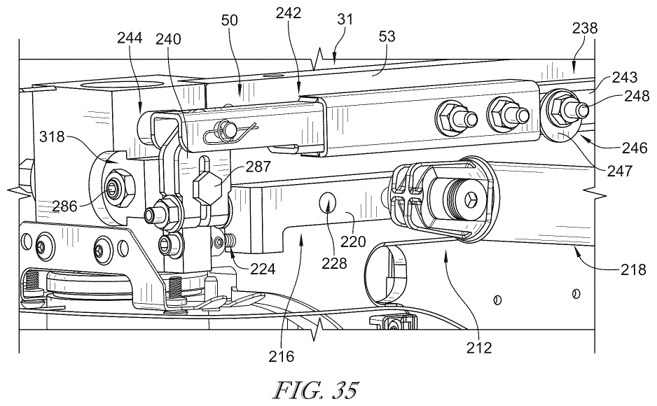

The mount 216 includes a mount body 220, one or more fasteners 222, and a jack screw 224 as shown in FIGS. 35-37. The fasteners 222 extend into the mount body 220 and the plate 53 or fixture on the lower plate 53 to couple the mount body 220 with the plate 53. The mount body 220 is pivotably coupled with the linear actuator 218 and provides a coupling feature to secure the linear actuator 218 with the lower plate 53. The jack screw 224 is coupled with the lower plate 53 or other fixture on the lower plate 53 and is configured to engage selectively the mount body 220 such as, for example, during a calibration of the brake system 50 as described in further detail below.

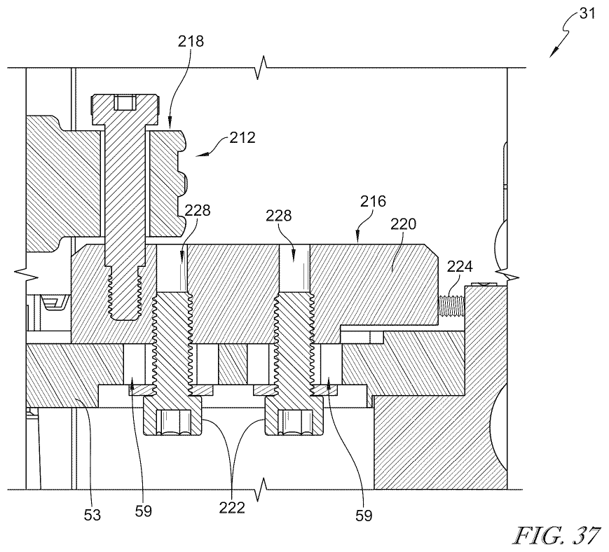

The mount body 220 is formed to define holes 228 for receiving the fasteners 222 and linear actuator bolt as shown in the section view of FIG. 37. The holes 228 are threaded in the illustrative embodiment. The lower plate 53 (or other fixture on the lower plate 53) is formed to include elongated slots 59 that extend through the lower plate 53. The fasteners 222 extend through the elongated slots 59 and into the mount body 220 to couple the mount body 220 with the lower plate 53. The elongated slots 59 allow the mount body 220 to move transversely relative to the lower plate 53 such as, for example, during the calibration process. The fasteners 222 are tightened to block the movement of the mount body 220 relative to the lower plate 53. In other embodiments, the mount body 220 is formed with elongated slots and/or the lower plate 53 is formed with threaded or unthreaded holes.

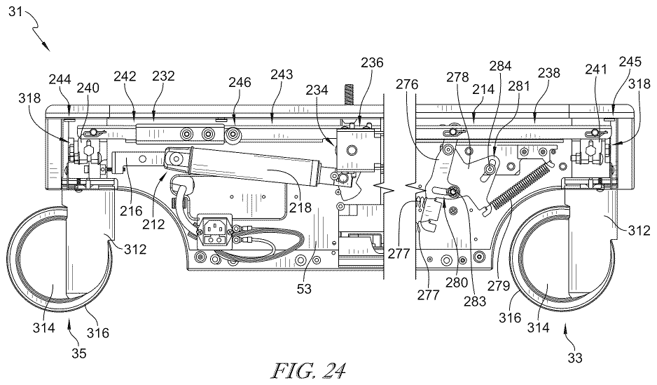

The casters 33, 35 each include a stem 312, a hub 314, and a wheel 316 as shown in FIG. 24. The stem 312 is coupled to the lower plate 53 of one of the columns 24, 26. The hub 314 and the wheel 316 are coupled to the stem 312 for pivotable movement about a vertical axis so that the caster can change direction relative to the base 31. The wheel 319 is coupled to the hub 314 for rotation about a horizontal axis so that the casters 33, 35 can roll along the floor 16.

Each of the four casters 33, 35 also include a braking element 318 shown, for example, in FIGS. 21, 35, and 36 and diagrammatically in FIG. 2. Each braking element 318 is movable between an unbraked and braked position. In the unbraked position, the braking element 318 allows pivoting of the hub 314 and the wheel 316 about the vertical axis and allows rotation of the wheel 316 about the horizontal axis. In the braked position, the braking element 318 blocks pivoting of the hub 314 and the wheel 316 about the vertical axis and blocks rotation of the wheel 316 about the horizontal axis, thereby completely immobilizing the casters 33, 35.

When the casters 33, 35 of the braking system 50 are driven to the braked position or the unbraked position by the remote pendant 60 or panel 61, the braking elements 318 of all four casters 33, 35 are engaged to immobilize the casters 33, 35 or disengaged to allow movement of the casters 33, 35. The immobilization of the casters 33, 35 prevents movement of the patient support apparatus 10 relative to the floor 16 during surgery. In the event of significant to total power loss to the patient support apparatus 10, the braking elements 318 of the casters 33, 35 can be moved to the unbraked position via a manual release assembly 236 included in the linkage 214.

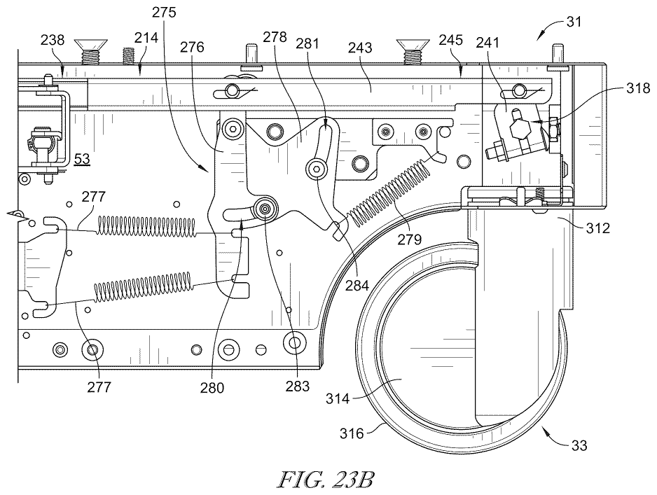

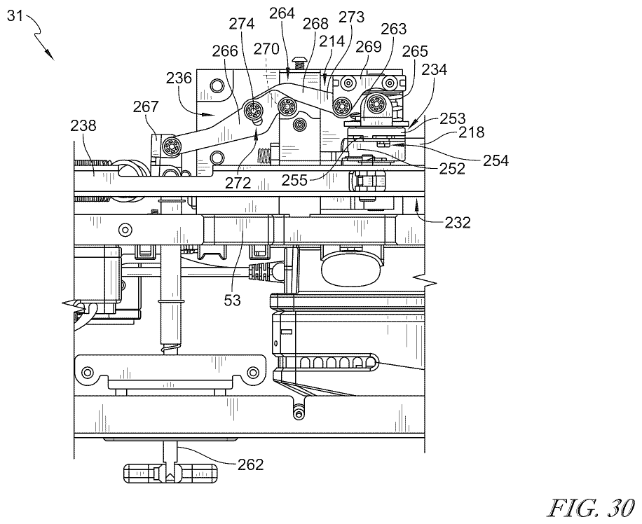

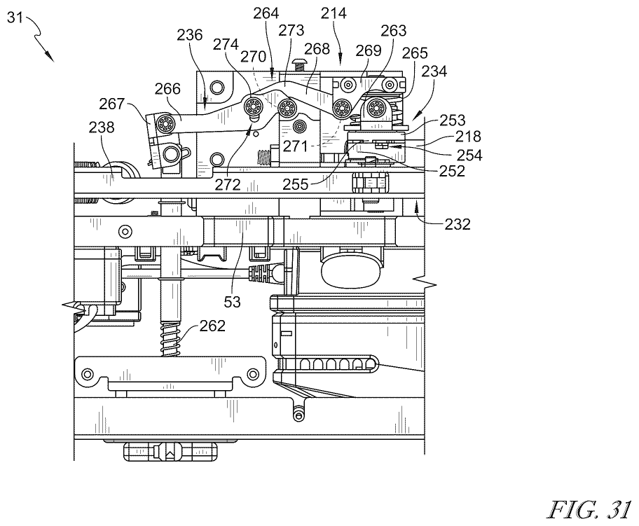

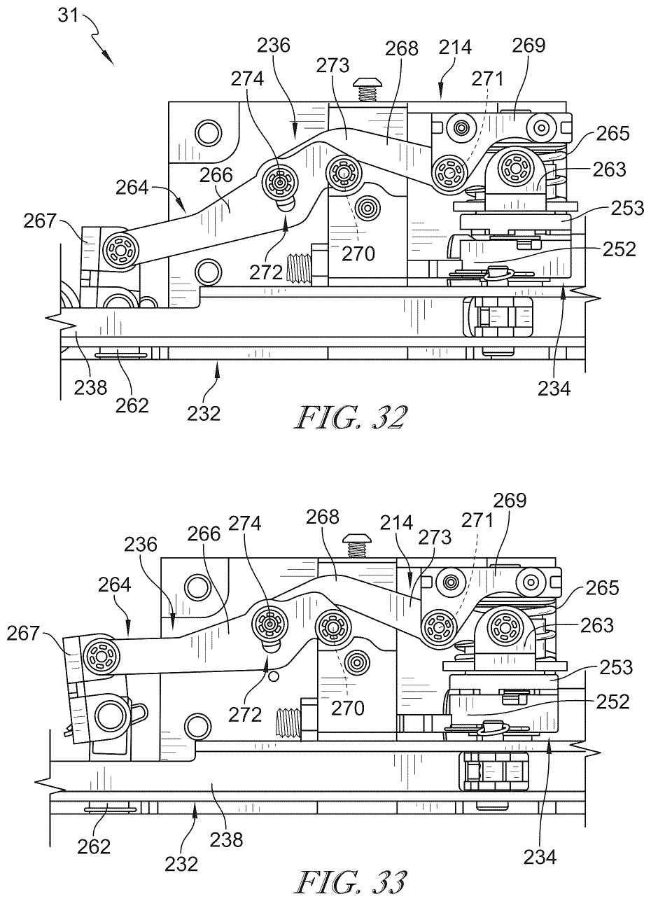

The linkage 214 is configured to convert linear motion from the brake drive 212 into rotation of the braking elements 318 so that the braking system 50 is driven between the braked and the unbraked positions as suggested in FIGS. 23 and 24. The linkage 214 is also configured to manually release the casters 33, 35 from the brake drive 212 and to move the braking elements 318 of the casters 33, 35 from the braked position and the unbraked position so that the braking system is in the unbraked position when a user manually releases the linkage 214 as suggested in FIGS. 30 and 32.

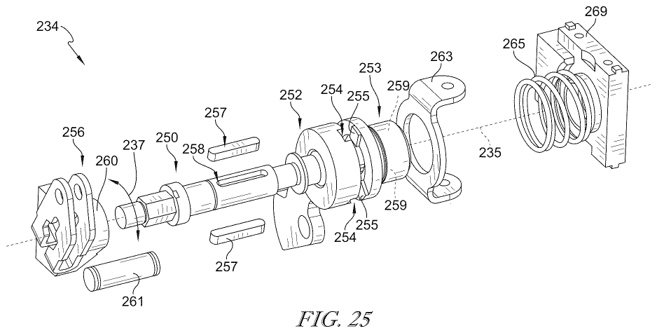

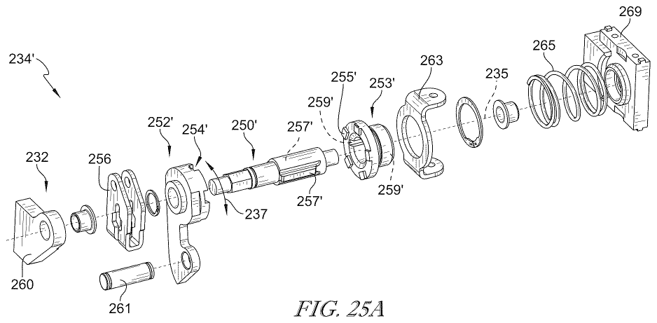

The linkage 214 includes a shaft assembly 232, a clutch assembly 234, and the manual release assembly 236 as shown in FIGS. 21-24. The shaft assembly 232 is coupled with the braking elements 318 for moving the braking elements 318 between the unbraked position and the braked position. The clutch assembly 234 is configured to be rotated about a clutch axis 235 relative to the lower plate 53 (or mount 216 coupled to the lower plate 53), as indicated by arrow 237 in FIG. 25, by the linear actuator 218 to move the shaft assembly 232 and, thus, the braking elements 318 between the unbraked position and the braked position. The manual release assembly 236 is coupled with the clutch assembly 234 and the shaft assembly 232 and configured to operably decouple the linear actuator 218 from the shaft assembly 232 and bias the shaft assembly 232 into moving the braking element to the unbraked position in response to a user manually activating the manual release assembly 236 as suggested in FIGS. 31 and 32.

The shaft assembly 232 of the linkage 214 includes a rod 238, a first fork 240, and a second fork 241 as shown in FIGS. 23 and 24. The rod 238 is coupled with the clutch assembly 234 and configured to move transversely relative to the lower plate 53 in response to extension and retraction of the linear actuator 218. The rod 238 may move along an arcuate path as a result of the rotation of the clutch assembly 234 with the rod 238 remaining generally horizontal to the floor 16 during its travel. The first fork 240 is pivotably coupled with the rod 238 and with the braking element 318 of a caster 35. The second fork 241 is pivotably coupled with the rod 238 and with the braking element 318 of a caster 33. Movement of the rod 238 causes the forks 240, 241 to rotate the braking elements 318 and move the braking elements 318 between the braked position and the unbraked position as shown in FIGS. 23 and 24. In the illustrative embodiment, the shaft assembly 232 moves both braking elements 318 of casters 33, 35 substantially simultaneously.

The rod 238 includes a first shaft 242 and a second shaft 243 coupled with the first shaft 242 as shown in FIGS. 23 and 24. The first shaft 242 and the first fork 240 cooperate to define a first caster brake link 244 and the second shaft 243 and the second fork 241 cooperate to define a second caster brake link 245. The first shaft 242 and the second shaft 243 are coupled together for movement together via fasteners during operation of the braking system 50. However, the first shaft 242 and the second shaft 243 are movable, selectively, relative to each other to adjust a length of the rod 238 for calibration. The first caster brake link and the second caster brake link and, therefore the rod 238, are biased toward moving the braking elements toward the unbraked position. The first shaft 242 and the second shaft 243 may each be made of a single component or multiple components coupled together, for example, by welding or fasteners.

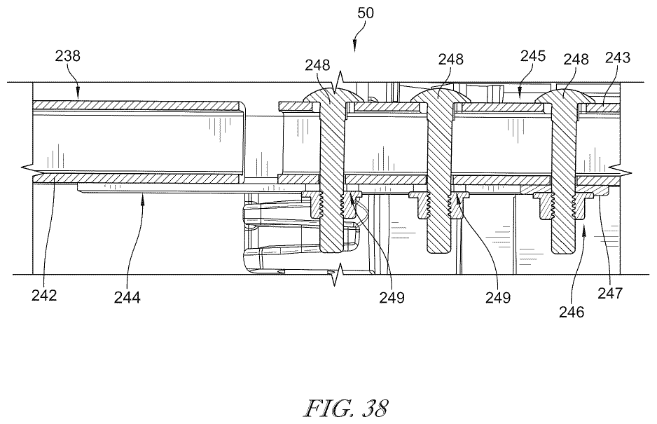

The shaft assembly 232 optionally includes a cam 246 coupled with one of the first caster brake link 244 and the second caster brake link 245 as shown in FIG. 35. The cam 246 is configured to engage the other of the first caster brake link 244 and the second caster brake link 245 to urge selectively the first caster brake link 244 away from the second caster brake link 245, for example, during calibration of the brake system 50. As shown in FIGS. 24 and 38, the cam 246 of the illustrated embodiment is coupled with the second shaft 243 of the second caster brake link 245 and may urge, selectively, the first shaft 242 and, thus, the first caster brake link 244 away from the second shaft 243. In the illustrative embodiment, the cam 246 includes a washer 247 with an eccentric profile that engages the first caster brake link 244 and a fastener 248 that couples the washer 247 with the second caster brake link 245. In other embodiments, the cam 246 may be provided by other suitable alternative components having a cam or wedge shape.

As shown in the section view of FIG. 38, one of the first caster brake link 244 and the second caster brake link 245 is formed to define one or more elongated slots 249. The shaft assembly 232 includes one or more fasteners 248 and one of the fasteners 248 extends through each of a corresponding one of the elongated slots 249 and into the other one of the first caster brake link 244 and the second caster brake link 245 to couple the first caster brake link 244 with the second caster brake link 245. As shown in FIG. 38, the first shaft 242 of the first caster brake link 244 is formed to include the elongated slots 249 and the second shaft 243 of the second caster brake link 245 is formed with circular holes. The elongated slots 249 assist in calibrating the brake system 50 as described further below.

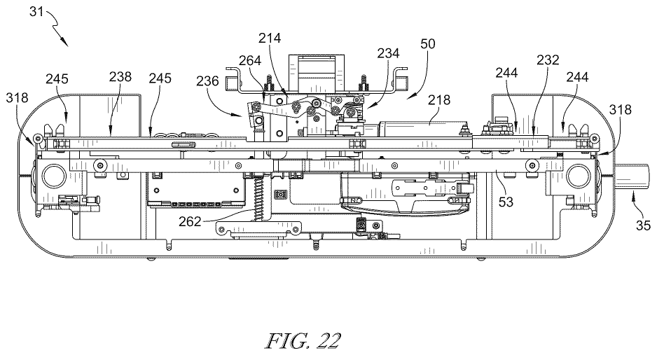

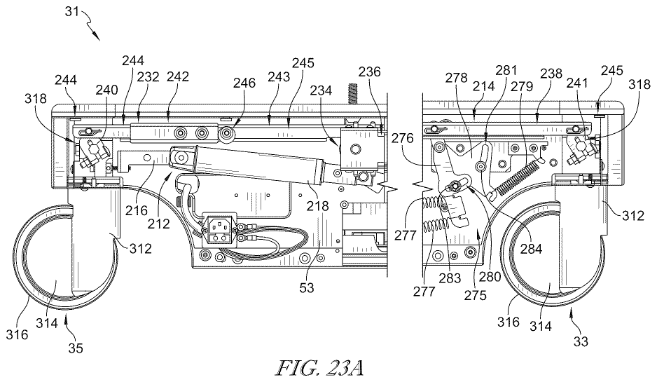

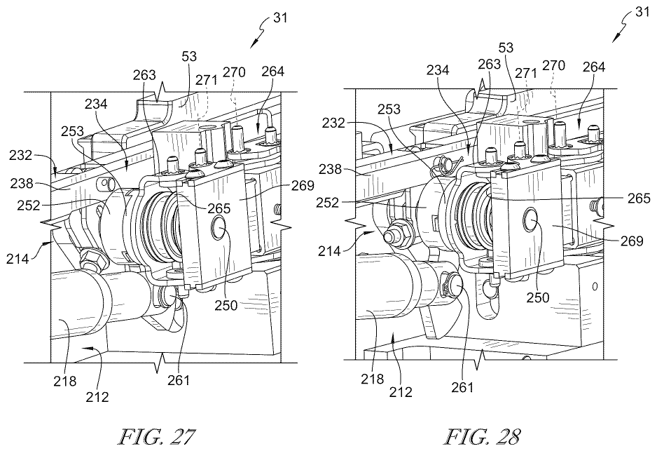

The clutch assembly 234 is coupled with the linear actuator 218 of the brake drive 212 and with the rod 238 of the linkage 214 as shown in FIGS. 22, 27, and 28. The clutch assembly 234 is movable between an engaged arrangement shown in FIGS. 27 and 28 and a disengaged arrangement shown in FIGS. 30 and 32. In the engaged arrangement, the clutch assembly 234 is rotatable by the linear actuator 218 to transmit motion from the linear actuator 218 through the shaft assembly 232 to the braking elements 318 to move the braking elements 318 between the unbraked position and the braked position. In the disengaged arrangement, clutch assembly 234 operably decouples the shaft assembly 232 from the linear actuator 218.

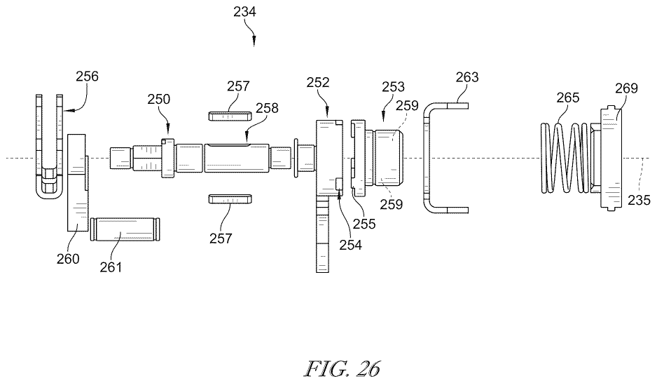

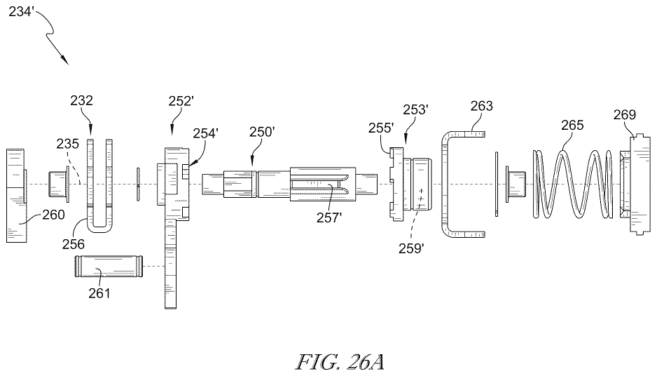

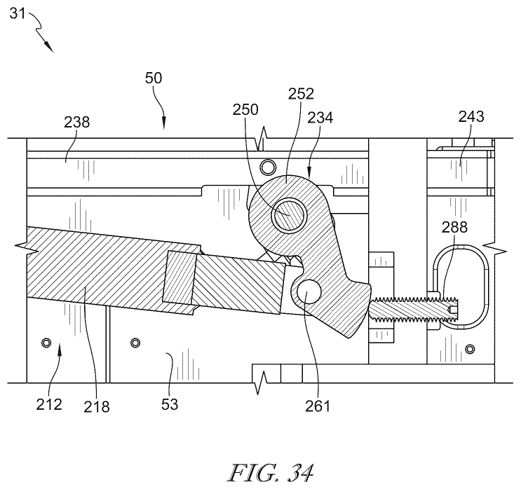

The clutch assembly 234 includes, among other things, a clutch shaft 250, a first clutch plate 252, and a second clutch plate 253 as shown in FIGS. 25 and 26. The clutch shaft 250 is rotatable about the clutch axis 235 and coupled to the shaft assembly 232 for movement therewith, as indicated by arrow 237 in FIG. 25. The clutch shaft 250 may be further supported or coupled with the manual release assembly 236. The first clutch plate 252 is coupled with the linear actuator 218 and arranged to rotate selectively about the clutch axis 235 relative to the clutch shaft 250. In the illustrative embodiment, the clutch shaft 250 provides a bearing surface for the clutch plate 252. In other words, the first clutch plate 252 cannot directly rotate the clutch shaft 250. The second clutch plate 253 is coupled for rotation with the clutch shaft 250. The first clutch plate 252 includes a body and a lever arm extending from the body. The lever arm is coupled with the linear actuator 218 via a hole formed in the lever arm. The lever arm includes an arm coupled with the linear actuator and a protrusion that extends from the arm and is configured to abut a travel stop 288 to limit movement of the linear actuator 218 as suggested in FIG. 34.

The second clutch plate 253 is movable between an engaged position to cause the clutch assembly 234 to be in the engaged arrangement and a disengaged position to cause the clutch assembly 234 to be in the disengaged arrangement. In the engaged position of the second clutch plate 253, the second clutch plate 253 is rotatably coupled with the first clutch plate 252 such that the clutch assembly 234 transmits motion from the linear actuator 218 through the first clutch plate 252, the second clutch plate 253, and then the clutch shaft 250 to the shaft assembly 232. In the disengaged position of the second clutch plate 253, the second clutch plate 253 is rotatably decoupled from the first clutch plate 252 such that the second clutch plate 253 and, thus, the shaft assembly 232 are free to move relative to the first clutch plate 252 and the linear actuator 218. The second clutch plate 253 is movable axially along the clutch axis 235 relative to the clutch shaft 250 between the engaged position and the disengaged position in the illustrative embodiment.

At least one of the first clutch plate 252 and the second clutch plate 253 is formed to define at least one recess 254 therein and the other of the first clutch plate and the second clutch plate is formed to include at least one tab 255 as shown in FIGS. 25 and 26. The at least one recess 254 may include a plurality of recesses 254 such as two, three, four, five, etc. recesses and the at least one tab 255 may include a plurality of tabs such as two, three, four, five etc. tabs. Illustratively, the body of the first clutch plate 252 is formed to include the at least one recess 254 and the number of recesses 254 is the same as the number of tabs 255. The tab 255 extends into the recess 254 to rotatably couple the first clutch plate 252 with the second clutch plate 253 in response to the second clutch plate 253 being in the engaged position as shown in FIGS. 27 and 28. The tab 255 is spaced from the recess 254 to rotatably decouple the second clutch plate 253 from the first clutch plate 252 in response to the second clutch plate 253 being in the disengaged position as shown in FIGS. 30 and 32. In some embodiments, each of the first clutch plate 252 and the second clutch plate 253 are formed to include both recesses 254 and tabs 255.

The clutch shaft 250 is coupled with the rod 238 of the shaft assembly 232 via a coupler 256 as shown in FIGS. 25 and 26. Bushings may be used with the clutch shaft 250 to support the clutch shaft 250. The bushings may be separate or integral with the clutch shaft 250. The coupler 256 is fixed with the clutch shaft 250 and pivotably coupled with the rod 238 to cause the rod 238 to move in response to rotation of the shaft 250. The clutch assembly 234 includes one or more keys 257 that interlocks the second clutch plate 253 with the clutch shaft 250 while allowing the second clutch plate 253 to slide selectively axially relative to the clutch shaft 250. The keys 257 are received in apertures 258 formed in the clutch shaft 250 in the illustrative embodiment to interlock the keys 257 with the clutch shaft 250. The second clutch plate 253 is formed to include slots 259 formed in the inner diameter of the second clutch plate 253. The slots 259 interlock with the keys 257 to couple the second clutch plate 253 with the clutch shaft 250 for radial movement while allowing the second clutch plate 253 to move axially relative to the clutch shaft into and out of engagement with the first clutch plate 252. In some embodiments, the keys 257 are integrally formed with one of the clutch shaft 250 and the second clutch plate 253 as shown in an example embodiment illustrated in FIGS. 25A and 26A. In some embodiments, the clutch shaft 250 is formed with slots 259 and the second clutch plate 253 is formed with apertures 258.

The clutch assembly 234 includes a mount plate 260 and a pin 261 as shown in FIGS. 25 and 26. The mount plate 260 is secured with the lower plate 53 and provides a bearing surface for the clutch shaft 250. The pin 261 extends into the first clutch plate 252 and the linear actuator 218 to couple the linear actuator 218 with the clutch plate 252. The brake drive 212 includes a travel stop 288 that is fixed relative to the mount 216, the lower plate 53, or another fixture on the lower plate 53 as shown in FIG. 34. The travel stop 288 is configured to limit a maximum extension of the linear actuator 218. In the illustrative embodiment, the travel stop 288 includes a fastener that extends through a support structure on the lower plate 53. A position of the terminal end of the fastener can be adjusted relative to the lower plate 53 to vary the allowed travel of the linear actuator 218. The travel stop 288 is configured to engage a portion of the first clutch plate 252 or the linear actuator 218 directly to block extension of the linear actuator beyond a predetermined location. As such, the travel stop 288 blocks the linear actuator 218 from overextending beyond a point that the linear actuator 218 would drive the clutch assembly 234 or the shaft assembly 232 beyond an operable point and damage or impair functionality of the brake system 50.

Another embodiment of a clutch assembly 234′ is shown in FIGS. 25A and 26A. The clutch assembly 234′ is substantially the same as the clutch assembly 234 described herein with notable differences shown and described herein. The clutch assembly 234′ includes among other things, a clutch shaft 250′, a first clutch plate 252′, and a second clutch plate 253′. The clutch shaft 250′ is rotatable about the clutch axis 235 and coupled to the shaft assembly 232 for movement therewith, as indicated by arrow 237. The first clutch plate 252′ is coupled with the linear actuator 218 and arranged to rotate selectively about the clutch axis 235 relative to the clutch shaft 250′. The second clutch plate 253′ is coupled for rotation with the clutch shaft 250′. The first clutch plate 252′ includes a body and a lever arm extending from the body. The first clutch plate 252′ is formed to define a plurality of recesses 254′ therein and the second clutch plate 253′ is formed to include a plurality of tabs 255′ as shown in FIGS. 25A and 26A. The plurality of recesses 254′ includes five recesses and the plurality of tabs 255′ includes five tabs. The tabs 255′ are configured to extend into the recesses 254′ to rotatably couple the first clutch plate 252′ with the second clutch plate 253′ in response to the second clutch plate 253′ being in the engaged position.

The clutch shaft 250′ is coupled with the rod 238 of the shaft assembly 232 via a coupler 256. Bushings may be used with the clutch shaft 250 to support the clutch shaft 250′. The clutch assembly 234′ includes keys 257′ that interlocks the second clutch plate 253′ with the clutch shaft 250′ while allowing the second clutch plate 253′ to slide selectively axially relative to the clutch shaft 250′. The keys 257′ are integrally formed with the clutch shaft 250′ as a single, one piece component as shown in FIGS. 25A and 26A. The second clutch plate 253′ is formed to include slots 259′ formed in the inner diameter of the second clutch plate 253′. The slots 259′ interlock with the keys 257′ to couple the second clutch plate 253′ with the clutch shaft 250′ for radial movement while allowing the second clutch plate 253′ to move axially relative to the clutch shaft into and out of engagement with the first clutch plate 252′. The clutch assembly 234′ further includes the mount plate 260 and the pin 261 as shown in FIGS. 25A and 26A.

In powered operation, the braking system 50 may be in the braked position or the unbraked position as shown in FIGS. 23A and 24. When in the braked position, the linear actuator 218 is in the extended position as shown in FIG. 23A. In response to a user input to remote pendant 60 or panel 61, the linear actuator 218 is retracted and rotates the first clutch plate 252 about the clutch axis 235. Because the second clutch plate 253 is engaged with the first clutch plate 252 from the bias force of bias member 265, the first clutch plate 252 transfers rotational motion through the second clutch plate 253 to the clutch shaft 250 via the keys 257. Rotation of the clutch shaft 250 causes the rod 238 of the shaft assembly 232 to move transversely via the coupler 256 and moves the braking elements 318 to the unlocked position. The rod 238 may move along an arcuate path in a first direction to unlock the braking elements 318.

To brake the braking elements 318 from the unlocked position, the linear actuator 218 retracts in response to a user input to remote pendant 60 or panel 61 to cause the first clutch plate 252 to rotate about the clutch axis 235 as indicated by arrow as suggested in FIGS. 24 and 25. Because the second clutch plate 253 is engaged with the first clutch plate 252 from the bias force of bias member 265, the first clutch plate 252 transfers rotational motion through the second clutch plate 253 to the clutch shaft 250 via the keys 257. Rotation of the clutch shaft 250 causes the rod 238 of the shaft assembly 232 to move transversely via the coupler 256 and moves the braking elements 318 to the locked position. The rod 238 may move along an arcuate path in a second direction to lock the braking elements 318.

The manual release assembly 236 includes a plunger 262, a clutch release fork 263, and a clutch release linkage 264 as shown in FIGS. 27-33. The plunger 262 is movable relative to the lower plate 53 and the mount 216 fixed with the lower plate 53 and configured to be moved by the user to activate the manual release to unbrake the braking elements 318 of casters 33, 35 on one of the columns 24, 26. The clutch release fork 263 is coupled with the clutch assembly 234 and the clutch release linkage 264 to move the second clutch plate 253 between the engaged and disengaged position. The clutch release linkage 264 is coupled with the plunger 262 and the clutch release fork 263 and configured to cause the clutch release fork 263 to move the clutch assembly 234 and operably decouple the linear actuator 218 from the shaft assembly 232 in response to the user pulling the plunger 262 or otherwise activating the plunger 262 or other suitable alternative.

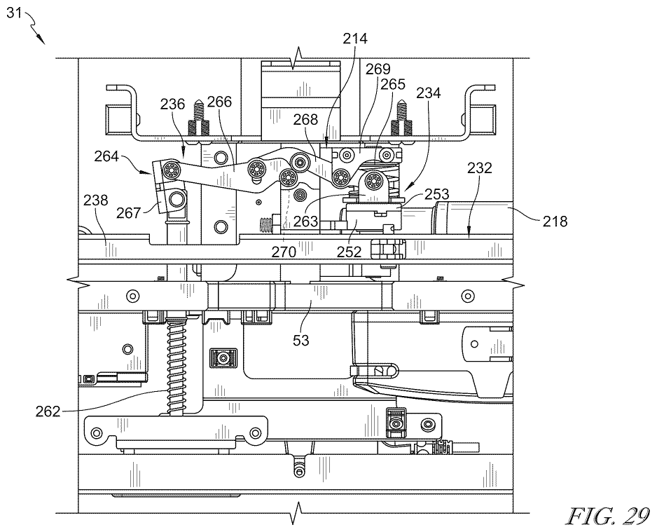

The plunger 262 extends through the cover 55 and the lower plate 53 of the base 31 such that a handle of the plunger 262 is accessible to the user from external the base 31. The plunger 262 includes a rod coupled with the handle and a bias member arranged around the rod and configured to bias the plunger 262 toward a retracted position wherein the handle is near the cover 55 as shown in FIG. 29. An end of the rod is pivotably coupled with the clutch release linkage 264.

The clutch release fork 263 is pivotably coupled with the clutch release linkage 264 and is coupled with the second clutch plate 253. The clutch release fork 263 moves selectively the second clutch plate 253 axially away from the first clutch plate 252 to rotatably decouple the first clutch plate 252 from the second clutch plate 253 in response to the user pulling the plunger 262 as suggested in FIGS. 30 and 32. The clutch release fork 263 may be coupled with the second clutch plate 253 via a retainer ring or other suitable alternative. The clutch assembly 234 includes a bias member 265 arranged around the clutch shaft 250 and engaged with the clutch release fork 263 and the clutch release linkage 264. The bias member 265 biases the clutch release fork 263 to urge the second clutch plate 253 into engagement with the first clutch plate 252.

The clutch release linkage 264 includes a first lever 266 pivotably coupled with the plunger 262 via a coupler link 267 and a second lever 268 pivotably coupled with the first lever 266 and with the clutch release fork 263 as shown in FIGS. 29-33. The clutch release linkage 264 further includes a support body 269 fixed relative to the lower plate 53 to support the second lever 268, clutch shaft 250, and the bias member 265. The first lever 266 is rotatable about a first lever axis 270 in response to movement of the plunger 262 relative to the lower plate 53. The second lever 268 is pivotably coupled with the first lever 266 and the release fork 263 and rotatable about a second lever axis 271. As such, pulling the plunger 262 causes the first lever 266 to rotate the second lever 268 about the second lever axis 271 and move the release fork 263 and the second clutch plate 253 away from the first clutch plate 252.

The first lever 266 and the second lever 268 may cooperate to provide some loss-motion to allow the plunger 262 to retract at least partway after being pulled. As a result, the plunger 262 extends out into the room less than its fully extending position while awaiting the brake system 50 to reengage the clutch assembly 234 after the manual release is activated. As shown in FIGS. 30 and 31, the first lever 266 is formed to include an elongated slot 272 and the second lever 268 includes a lever body 273 and a pin 274 that extends through the lever body 273 and the elongated slot 272 to couple the second lever 268 with the first lever 266. The elongated slot 272 allows for limited movement of the first lever 266 relative to the second lever 268 to allow the plunger 262 to be returned toward an initial position after being pulled while the second clutch plate 253 is disengaged with the first clutch plate 252.

As an example, FIG. 31 shows the manual release assembly 236 after the plunger 262 has been pulled to disengage the second clutch plate 253 from the first clutch plate 252 and then the plunger 262 has been released such that it has moved a first distance back toward its initial position due to bias member 265 urging the second clutch plate 253 toward the first clutch plate 252 (while still being disengaged from the first clutch plate 252). The elongated slot 272 allows the first lever 266 to further rotate from its position in FIG. 31 relative to the second lever 268 and thereby allow the plunger 262 to move further toward the lower plate 53 while the second lever 268, clutch release fork 263, and second clutch plate 253 are maintained in their positions.

The manual release assembly 236 includes a brake-release lever assembly 275 configured to urge the shaft assembly 232 into moving the braking elements 318 toward the unbraked position as suggested in FIGS. 23A, 23B, and 24. The brake-release lever assembly 275 may dampen the movement of the shaft assembly 232 so that the brake system 50 operates without strong forces and hard stops. The brake-release lever assembly 275 is coupled with the shaft assembly 232 and the lower plate 53 of the base 31 of the patient support apparatus 10.

The brake-release lever assembly 275 includes a first brake-release lever 276 and at least one first bias member 277 as shown in FIGS. 23A and 24. The first brake-release lever 276 is pivotably coupled with the lower plate 53 of the base 31 and with the second shaft 243 of the shaft assembly 232 and configured to rotate about a first brake-release lever axis. The first bias member 277 is coupled with the first brake-release lever 276 and the lower plate 53 of the base 31 to bias the first brake-release lever 276 such that the first brake-release lever 276 urges the shaft assembly 232 into moving the braking elements 318 toward the unbraked position. In the illustrative embodiment, the at least one first bias member 277 includes two bias members. In embodiments with only one first bias member 277, the upper first bias member 277 may be the bias member that is omitted. When the clutch assembly 234 is in the engaged arrangement and the brake elements 318 are in the braked position as shown in FIG. 23A, the bias member 277 urges the first brake-release lever 276 to move the shaft assembly 232 toward unlocking the braking elements 318; however, the bias member 277 does not overcome the linear actuator 218 such that the braking elements 318 remain braked. In response to the clutch assembly 234 being moved the disengaged arrangement by the manual release 236, the shaft assembly 232 is operably decoupled from the linear actuator 218 and the bias member 277 urges the first brake-release lever 276 and, thus, the shaft assembly 232 to move the braking elements 318 to the unlocked position to unlock the casters 33, 35.

The brake-release lever assembly 275 may include a second brake-release lever 278 and a second bias member 279 to dampen or regulate the speed of the first brake-release lever 276 during a manual release event. As such, a hard stop or fast deacceleration of the first brake-release lever 276 may be avoided. The second brake-release lever 278 is pivotably coupled with the lower plate 53 of the base 31 to rotate about a second brake-release lever axis. The second brake-release lever 278 is coupled with the first brake-release lever 276 for limited relative movement between the two levers 276, 278. The second bias member 279 is coupled with the lower plate 53 of the base 31 and with the second brake-release lever 278 to cause the second brake-release lever 278 to dampen movement of the first brake-release lever 276. The second brake-release lever 278 and the second bias member 279 bias the first brake-release lever 276 in an opposition direction as the first bias member 277. The second bias member 279 applies a smaller moment onto the first brake-release lever 276 via the second brake-release lever 278 as compared to the at least one first bias member 277.

The first brake-release lever 276 is formed to include a first curved elongated slot 280 and the second brake-release lever 278 is formed to include a second curved elongated slot 281 as shown in FIGS. 23A, 23B, and 24. The brake-release lever assembly 275 further includes a first pin 283 extending through the first curved elongated slot 280 and the second brake-release lever 278 and a second pin 284 extending through the second curved elongated slot 281 and into the lower plate 53 of the base 31. The first pin 283 extends through a circular hole in the second brake-release lever 278 so that the pin 283 moves with the second brake-release lever 278.

When the braking elements 318 are locked as shown in FIG. 23A, the at least one first bias member 277 urges the first brake-release lever 276 toward unlocking the braking elements 318 as described above. The second bias element 278 urges the second brake-release lever 278 in an opposite direction of the first brake-release lever 276 until the second pin 284 engages a terminal end of the second curved elongated slot 281 to block further movement of the second brake-release lever 278. The first pin 283 is between terminal ends of the first curved elongated slot 280 so that the first brake-release lever 276 may rotate a limited amount relative to the second brake-release lever 278 before engaging the second brake-release lever 278.

When the braking elements 318 are in the process of being moved from locked to unlocked due to a manual release, the brake-release lever assembly 276 moves the braking elements 318 with a dampened action as suggested in FIG. 23B. In response to the clutch assembly 234 being disengaged by the manual release assembly 236, the first brake-release lever 276 is rotated by the at least one bias member 277, illustratively in the clockwise direction, to cause the rod 238 of the shaft assembly 232 to move transversely and unlock the braking elements 318. The first brake-release lever 276 rotates a first amount relative to the second brake-release lever 278 until the first pin 283 reaches the terminal end of the slot 280 and engages the first brake-release lever 276. Thereafter, the second brake-release lever 278 applies a resistive or dampening force onto the first brake-release lever 276 caused by the second bias element 279. In other words, the rotation of the first brake-release lever 276 and, thereby, the speed of the rod 238 is reduced by the counteracting force of the second bias member 279. As shown in FIG. 23B, the second pin 284 may be located near a first terminal end of the second slot 281 during an initial timing of the unlocking motion.

When the braking elements 318 are in the unlocked position as shown in FIG. 24, the first brake-release lever 276 is biased counterclockwise. The first pin 283 engages the first brake-release lever 276 such that the second brake-release lever 278 applies the counteracting force from the second bias member 279 onto the first brake-release lever 276. Again, the force of the second bias member 279 is less than the force of the at least one first bias member 277 and is configured to dampen the movement of the first brake-release lever 276. The second pin 284 may be located partway between terminal ends of the second slot 281 in response to the braking elements 318 being in the unlocked position.

To manually release the casters 33, 35 from the brake drive 212 when the brake system 50 is in the braked position, a user pulls the plunger 262 as suggested in FIGS. 29 and 30. In response to the plunger 262 being pulled out, the coupler link 267 causes the first lever 266 to rotate about the axis 270. The pin 274 engages the second lever 268 to cause the second lever 268 to rotate about the axis 271 and move the clutch fork 263 to move the second clutch plate 253 along the clutch axis 235 away from the first clutch plate 252 as shown in FIG. 32. The second clutch plate 253 is spaced apart from the first clutch plate 252 such that the tabs 255 are moved out of the recesses 254. As a result, the linear actuator 218 is operably decoupled from the shaft assembly 232. The force of the least one bias element 277 urges the first brake-release lever 276 to rotate and move the rod 238 of the shaft assembly 232 to unlock the braking elements 318. The optional second brake-release lever 278 and second bias member 279 dampen the movement of the first brake-release lever 276. Movement of the rod 238 causes the clutch shaft 250 and second clutch plate 253 to rotate about the clutch axis 235 as suggested in FIG. 30 by the misaligned tabs 255 and recesses 254.

In response to the user releasing the plunger 262, the bias member arranged around the plunger 262 causes the plunger 262 to move toward its initial position as suggested in FIG. 31. As a result, the first lever 266 and the second lever rotate partway and allow the bias member 265 to urge the second clutch plate 253 toward the first clutch plate 252. However, the tabs 255 of the second clutch plate 253 are misaligned with the recesses 254 such that the second clutch plate 253 is not in the engaged arrangement with the first clutch plate 252 as suggested in FIG. 31. This contact blocks the manual release assembly 236 from fully returning to it initial unactivated arrangement of FIG. 29. In the illustrative embodiment, the slot 272 allows the plunger 262 to move further towards its initial position while the clutch plates 252, 253 remain in contact, but disengaged, by allowing the first lever 266 to rotate an additional limited amount about the axis 270 without rotating the second lever 268.

To engage the clutch plates 252, 253 to allow powered braking and unbraking after a manual release event, the linear actuator 218 is retracted in response to a user input to remote pendant 60 or panel 61 to cause the first clutch plate 252 to rotate about the clutch axis 235 relative to the second clutch plate 253 until the tabs 255 are aligned circumferentially with the recesses 254. Once aligned, the bias member 265 automatically urges the second clutch plate 253 axially relative to the clutch axis 235 into the engaged position in which the tabs 255 are received in the recesses 254 as suggested in FIG. 29. The plunger 262, first lever 266, and second lever 268 are returned to their initial position and ready for another manual release. Once the clutch assembly 234 is reengaged, any further retracting or extension of the linear actuator 218 results in powered locking or unlocking of the braking elements 318.

A method of synchronizing the brake system 50 and the braking elements 318 of the patient support apparatus 10 is hereafter described with reference to FIGS. 35-38. With the fasteners 248 that extend through the first caster brake link 244 and the second caster brake link 245 loosened, the first caster brake link 244 is moved to a predetermined position to cause the first braking element 318 of the caster 35 to be unlocked and the second caster brake link 245 is moved relative to the first caster brake link 244 to a predetermined position to cause a second braking element 318 of the caster 33 to be unlocked as suggested in FIG. 24.

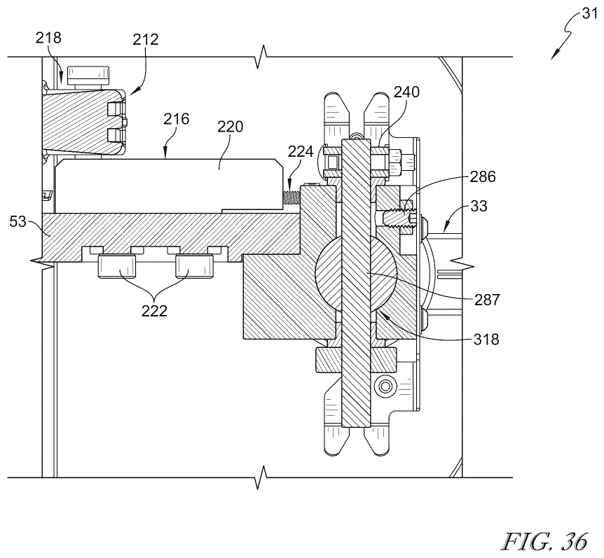

Relative movement of the first caster brake link 244 and the second caster brake link 245 away from the predetermined positions, is temporarily restricted. In the illustrative embodiment, set screws 286 are tightened and used to temporarily restrict rotation of a hex bolt 287 to block movement of the first and second caster brake links 244, 245. As suggested in FIGS. 35 and 36, a first set screw 286 is tightened relative to the lower plate 53 of the base 31 of the patient support apparatus 10 to engage the hex bolt 287 of the first braking element 318 to block rotation of the hex bolt 287 and thereby retain the first caster brake link 244 in the predetermined position. A second set screw 286 is tightened relative to the base 31 of the patient support apparatus to engage the hex bolt 287 of the second braking element 318 to block rotation of the hex bolt 287 and thereby retain the second braking element 318 in the predetermined position.

With the linear actuator 218 in the retracted position, a force is applied to the actuator mount 216 to urge the actuator mount 216 away from the caster 35 and toward the other of caster 33. The slotted holes 59 formed in the lower plate 53 of the base 31 allow the actuator mount 216 to move relative to the lower plate 53. The actuator mount 216 is then fixed with the lower plate 53 of the base 31 of the patient support apparatus 10 by tightening the fasteners 222 while the force is applied to the actuator mount 216 as suggested in FIG. 37. In the illustrative embodiment, the jack screw 224 extends through the base 31 of the patient support apparatus 10 and engages the actuator mount 216 to apply the force and urge the actuator mount 216 away from the caster 35.

A force is applied to the first caster brake link 244 and the second caster brake link 245 to urge the first caster brake link 244 away from the second caster brake link 245 while the first and second braking element 318 remain unlocked due to the set screws 286. In particular, the cam 246 is rotated to cause the washer 247 to engage the first caster brake link 244 and urge the first caster brake link 244 away from the second caster brake link 245 as suggested in FIG. 35.

The first caster brake link 244 is fixed with the second caster brake link 245 for movement with the second caster brake link 245 by tightening the other fasteners 248 while the force of the cam 246 remains applied to the first caster brake link 244 and the second caster brake link 245. After the first and second brake links 244, 245 are fixed together via the fasteners 248, the set screws 286 are backed away from engagement with the hex bolts 287 to unrestrict the movement of the first and second caster brake links 244, 245. As such, the first and second caster brake links 244, 245 are free to move together between locked and unlocked positions to lock and unlock the braking elements 318. The jack screw 224 may remain in position or may be rotated to be moved away from the mount 216.

The foregoing description of various embodiments and principles of the disclosure have been presented for the purposes of illustration and description. It is not intended to be exhaustive or to limit the disclosure to the precise forms disclosed. Many alternatives, modifications and variations will be apparent to those skilled in the art. Moreover, although multiple inventive aspects and principles have been presented, these need not be utilized in combination, and various combinations of inventive aspects and principles are possible in light of the various embodiments provided above. Accordingly, the above description is intended to embrace all possible alternatives, modifications, aspects, combinations, principles, and variations that have been discussed or suggested herein, as well as all others that fall within the principles, spirit and broad scope of the various possible inventions disclosed herein and defined by the claims.

1. A patient support apparatus comprising:
a caster including a stem, a wheel coupled to the stem to rotate about a wheel axis relative to the stem, and a braking element movable from an unbraked position allowing rotation of the wheel relative to the stem to a braked position blocking the wheel from rotation relative to the stem,
a brake drive including a mount and a linear actuator coupled to the mount and movable between a retracted position and an extended position relative to the mount, and
a linkage configured to transfer motion of the linear actuator to the braking element to move the braking element between the unbraked position and the braked position, the linkage includes a shaft assembly coupled with the braking element, a clutch assembly configured to be rotated about a clutch axis relative to the mount by the linear actuator to move the braking element between the unbraked position and the braked position via the shaft assembly, and a manual release assembly coupled with the clutch assembly and the shaft assembly and configured to operably decouple the linear actuator from the shaft assembly and bias the shaft assembly into moving the braking element to the unbraked position in response to a user manually activating the manual release.

2. The patient support apparatus of claim 1, wherein the clutch assembly is movable between an engaged arrangement in which the clutch is rotatable by the linear actuator to transmit motion from the linear actuator through the shaft assembly to the braking element to move the braking element between the unbraked position and the braked position and a disengaged arrangement in which the shaft assembly is operably decoupled from the linear actuator.

3. The patient support apparatus of claim 1, wherein the clutch assembly includes a clutch shaft rotatable about the clutch axis and coupled to the shaft assembly for movement therewith, a first clutch plate coupled with the linear actuator and arranged to rotate selectively about the clutch axis relative to the clutch shaft, and a second clutch plate coupled for rotation to the clutch shaft and movable between an engaged position in which the second clutch plate is rotatably coupled with the first clutch plate such that the clutch assembly transmits motion from the linear actuator through the first clutch plate, the second clutch plate, and the clutch shaft to the shaft assembly and a disengaged position in which the second clutch plate is rotatably decoupled from the first clutch plate such that the second clutch plate and the shaft assembly are free to move relative to the first clutch plate and the linear actuator, and the second clutch plate is movable axially along the clutch axis relative to the clutch shaft between the engaged position and the disengaged position.

4. The patient support apparatus of claim 3, wherein one of the first clutch plate and the second clutch plate is formed to define a recess therein and the other of the first clutch plate and the second clutch plate is formed to include a tab that extend into the recess to rotatably couple the first clutch plate with the second clutch plate in response to the second clutch plate being in the engaged position and that is spaced from the recess to rotatably decouple the second clutch plate from the first clutch plate in response to the second clutch plate being in the disengaged position.

5. The patient support apparatus of claim 2, wherein the clutch further includes a key that interlocks the second clutch plate with the clutch shaft while allowing the second clutch plate to slide axially relative to the clutch shaft.

6. The patient support apparatus of claim 1, wherein the manual release assembly includes a plunger movable relative to the mount, a clutch release fork coupled with the clutch assembly, and a clutch release linkage coupled with the plunger and the clutch release fork and configured to cause the clutch release fork to move the clutch assembly and operably decouple the linear actuator from the shaft assembly in response to the user pulling the plunger.

7. The patient support apparatus of claim 6, wherein the clutch release fork is coupled with the second clutch plate and moves selectively the second clutch plate axially away from the first clutch plate to rotatably decouple the first clutch plate from the second clutch plate in response to the user pulling the plunger, and the clutch release fork is biased to urge the second clutch plate into engagement with the first clutch plate.

8. The patient support apparatus of claim 6, wherein the clutch release linkage includes a first lever pivotably coupled with the plunger and rotatable about a first lever axis and a second lever pivotably coupled with the first lever and the clutch release fork and rotatable about a second lever axis such that pulling the plunger causes the first lever to rotate the second lever about the second lever axis and move the clutch release fork and the second clutch plate away from the first clutch plate.

9. The patient support apparatus of claim 8, wherein the first lever is formed to include an elongated slot and the second lever includes a lever body and a pin that extends through the lever body and the elongated slot to couple the second lever with the first lever and wherein the elongated slot allows for limited movement of the first lever relative to the second lever to allow the plunger to be returned toward an initial position after being pulled while the second clutch plate is disengaged with the first clutch plate.

10. The patient support apparatus of claim 1, further comprising a base and wherein the manual release assembly includes a brake-release lever assembly coupled with the shaft assembly and the base of the patient support apparatus and configured to urge the shaft assembly into moving the braking element toward the unbraked position.

11. The patient support apparatus of claim 10, wherein the brake-release lever assembly includes a first brake-release lever and a first bias member, the first brake-release lever is pivotably coupled with the base and with the shaft assembly and configured to rotate about a first brake-release lever axis, the first bias member is coupled with the first brake-release lever and the base to bias the first brake-release lever such that the first brake-release lever urges the shaft assembly into moving the braking element toward the unbraked position.

12. The patient support apparatus of claim 11, wherein the brake-release lever assembly further includes a second brake-release lever and a second bias member, the second brake-release lever is pivotably coupled with the base to rotate about a second brake-release lever axis and coupled with the first brake-release lever, the second bias member is coupled with the base and with the second brake-release lever to cause the second brake-release lever to dampen movement of the first brake-release lever, and the first brake-release lever includes a first curved elongated slot and the second brake-release lever includes a second curved elongated slot and the brake-release lever assembly further includes a first pin extending through the first curved elongated slot and the second brake-release lever and a second pin extending through the second curved elongated slot and into the base.

13. The patient support apparatus of claim 1, further comprising a second caster having a second wheel and a second braking element movable between an unbraked position and a braked position, the shaft assembly includes a first caster brake link coupled with the first braking element, a second caster brake link coupled with the second braking element and operably coupled with the linear actuator, and a cam coupled with one of the first caster brake link and the second caster brake link, the first caster brake link is movable selectively relative to the second caster brake link, and the cam is configured to engage the other of the first caster brake link and the second caster brake link to urge selectively the first caster brake link away from the second caster brake link.

14. The patient support apparatus of claim 1, further comprising a caster brake status indicator system that illuminates a light indicative of the braking element being in the unbraked position or the braked position, the caster brake status indicator system illuminates the light onto a floor underlying the patient support apparatus, and the light is a first color in response to the braking system being in the unbraked position and the light is a second color different from the first color in response to the braking system being in the braked position.

15. The patient support apparatus of claim 1, wherein the brake drive includes a travel stop that is fixed relative to the mount and the travel stop is configured to limit a maximum extension of the linear actuator.

16. The patient support apparatus of claim 1, further including a support base, a first lift column and a second lift column spaced apart from the first lift column, and a patient support extending between and coupled with the first lift column and the second lift column, the first lift column and the second lift column supported by the support base, the patient support being rotatable about an axis that extends between the first lift column and the second lift column, and a height of the first lift column and a height of the second lift column are adjustable to locate the axis in a horizontal orientation above a floor underlying the support base by a vertical distance, the vertical distance having a minimum value of equal to or less than 900 millimeters.

17. The patient support apparatus of claim 16, further comprising a remote pendant removably coupled with one of the first column and one of the second column and a panel fixed to one of the first lift column and the second lift column, each of the remote pendant and the panel having a plurality of buttons thereon, a height of the first lift column and the height of the second lift column are adjustable to locate the axis in a horizontal orientation above a floor underlying the support base by a vertical distance, and the buttons of the remote pendant and the buttons of the panel are each configured to adjust the height of the first lift column, the height of the second lift column, and rotation of the patient support about this axis.

18. A method of operating a brake system on a patient support apparatus, the method comprising:
extending a linear actuator to rotate a clutch about an axis and cause a shaft assembly to move a caster brake of a caster to a braked position and block rotation of a wheel of the caster,
disengaging a first clutch plate from a second clutch plate of the clutch assembly to operably decouple the shaft assembly from the linear actuator, and
moving the shaft assembly automatically in response to disengaging the first clutch plate from the second clutch plate to cause the shaft assembly to unbrake the caster brake.

19. The method of claim 18, further comprising retracting the linear actuator to rotate the first clutch plate and moving the second clutch plate into engagement with the first clutch plate to couple the first clutch plate with the second clutch plate after moving the shaft assembly automatically.

20. The method of claim 18, wherein disengaging the first clutch plate from the second clutch plate includes moving a plunger operably coupled with the second clutch plate from a first position to a second position, and the method further includes moving the plunger from the second position toward the first position without engaging the second clutch plate with the first clutch plate before retracting the linear actuator.
